(19) United States
(12) Patent Application Publication
(10) Pub. No.: US 2002/0081606 A1
(43) Pub. Date: Jun. 27, 2002

(54) Methods for detecting and identifying a gram positive bacteria in a sample

(75) Inventors: Patrick Trieu-Cuot, Fortenay aux Roses (FR); Clare Poyart, Fortenay aux Roses (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris Cedex (FR)

(21) Appl. No.: 09/860,432

(22) Filed: May 21, 2001

Related U.S. Application Data
(60) Provisional application No. 60/205,237, filed on May 19, 2000.

(51) Int. Cl. C12Q 1/68; C12P 19/34

(52) U.S. Cl. 435/6; 435/91.2

(57) ABSTRACT

The present invention provides fragments of a sodA gene from gram positive bacteria, methods of using these fragments as probes to detect and identify microorganisms in a sample and kits containing suitable reagents to perform the method.

### DESCRIPTION OF THE INVENTION

The present invention provides fragments of a sodA gene from gram positive bacteria, methods of using these fragments as probes to detect and identify microorganisms in a sample and kits containing suitable reagents to perform the methods.

### BACKGROUND OF THE INVENTION

Enterococci, although not highly virulent microorganisms, have emerged worldwide in the last decade as one of the leading causes of nosocomial bacteremia, surgical wound infections, and urinary tract infections (9, 10, 13, 24). This evolution is mainly due to the appearance of multiresistant strains of enterococci that can be resistant to most antibiotics used for the treatment (ampicillin, aminoglycosides, and glycopeptides). Most human enterococcal infections (≧90%) are caused by Enterococcus faecalis and Enterococcus faecium, however, the incidence of other species, such as Enterococcus casseliflavus and Enterococcus gallinarum, could be underestimated because of bacterial mis-identification. In clinical laboratories, accurate identification of enterococcal species is required to carry out a proper epidemiologic surveillance and may help in the management of infected patients in case of relapse. This is usually done by testing tolerance to bile esculine and tellurite, growth in 6.5% NaCl broth, specific carbohydrate utilization (2, 6), by characterizing bacterial motility and pigment production (1), and by using commercial biochemical test systems, such as the API-20 STREP or rapid ID 32 Strep. However, these phenotypic methods are often not reliable and the automated systems, such as the Vitek and MicroScan systems, do not properly identify enterococci other than E. faecalis and E. faecium in absence of additional tests (11). Consequently, several genotypic methods based on the analysis of PCR products derived from selected target DNA have been developed for species identification of enterococci (3, 14, 22). This includes the determination of the 16S rDNA sequence (18), a strategy which is now greatly facilitated by the use of universal 16S PCR primers associated with the development of simplified, partially automated, and cost effective sequencing technologies. However, the interpretation of these data may be complicated by the fact that divergent 16S rDNA sequences may exist within a single organism (23) or, alternatively, that closely related species may have identical 16S rDNA sequences (8), as recently shown in the genera Enterococcus for E. casseliflavus and E. gallinarum (18). To solve this problem, it is possible to use alternative monocopy target sequences which exhibit a higher divergence than that of the 16S rDNA. The sodA gene of the gram positive cocci which encodes the manganese-dependent superoxide dismutase fulfills these criteria and we recently reported that sequencing of the sodA PCR product with the use of a single pair of degenerate primers constitutes a valuable approach to the genotypic identification of the 29 streptococcal species (20). In this work, the same universal primers (19) were used to construct a sodA database of 19 enterococcal species including E. casseliflavus and E. gallinarum.

### BRIEF DESCRIPTION OF THE DRAWINGS

FIG. 1: Phylogenetic unrooted tree showing the relationships among the sodAᵢₙₜ fragments from various enterococcal type strains. The tree was established from an analysis of the sequences listed in Table 1 by using the neighbor-joining method. The sodAᵢₙₜ sequences of L. lactis, L. garvieae, S. bovis, S. pyogenes type strains were included in this work. The value on each branch is the estimated confidence limit (expressed as a percentage) for the position of the branch as determined by bootstrap analysis. Only the bootstrap values superior to 95%, which were considered as significant, are indicated. The scale bar (NJ distance) represents 10% differences in nucleotide sequences.

### DETAILED DESCRIPTION OF THE INVENTION

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

Fragments from sodA genes from a number of Enterococcus species are shown in SEQ ID NOS:1-19 and 21-36, from Lactococcus garvieae is shown in SEQ ID NO:20, from a number of Streptococcus species are shown in SEQ ID NOS:37-50, from a number of Abiotrophia species are shown in SEQ ID NOS:51-53, from a number of Staphlococcus species are shown in SEQ ID NOS:54-93 and from Macrococcus caseolyticus is shown in SEQ ID NO:94.

Microbial specimens for use in this invention can be obtained from any source suspected of harbouring bacteria. The samples are generally dispersed in a measured amount of buffer, though dispersal may be optimal if lysis is immediately possible. This dispersal buffer generally provides a biologically compatible solution. Samples may be frozen or used directly after obtaining.

Prior to analysis, samples suspected of containing bacteria are preferably subjected to a lysing solution to release cellular nucleic acids. Dispersal of the sample prior to lysis is optional. Lysing buffers are known in the art. Ausebel et al (eds), Current Protocols in Molecular Biology, John Wiley and Sons, Inc., 2000. Generally, these buffers are between pH 7.0 and 8.0, and contain both chelating agents and surfactants. Typically, a lysing solution is a buffered detergent solution having a divalent metal chelator or a buffered chaotrophic salt solution containing a detergent (such as SDS), a reducing agent and a divalent metal chelator (EDTA). The use of enzymes such as N-acetyl-muramidase (lysozyme) or proteases (such as Protease K) will facilitate lysis and offer high quality results.

The sample may be directly immobilized to a support or further processed to extract nucleic acids prior to immobilization. Released or extracted bacterial nucleic acid (including target nucleic acid) are fixed to a solid support, such as cellulose, nylon, nitrocellulose, diazobenzyloxymethyl cellulose, and the like. The immobilized nucleic acid can then be subjected to hybridization conditions.

Alternatively, samples may be collected and dispersed in a lysing solution that also functions as a hybridization solution, such as 3M guanidinium thiocyanate (GuSCN), 50 mM Tris (pH 7.6), 10 mM EDTA, 0.1% sodium dodecylsulfate (SDS), and 1% mercaptoethanol (Maniatis, T. et al. Molecular Cloning: A Laboratory Manual, Cold Spring Harbor, N.Y., 1982).

Alternatively, the nucleic acid probes may be immobilized onto solid phase microchips according to methods known in the art and subsequently hybridization with sample nucleic acids can be identified with a microchip reader. This and other solid phase microchip methods are disclosed in Ausebel et al (eds), Current Protocols in Molecular Biology, John Wiley and Sons, Inc., 2000.

Various degrees of stringency of hybridization can be employed. As the conditions for hybridization become more stringent, there must be a greater degree of complementarity between the probe and the target for duplex formation to occur. The degree of stringency can be controlled by temperature, ionic strength, pH and the presence of a partially denaturing solvent such as formamide. For example, the stringency of hybridization is conveniently varied by changing the polarity of the reactant solution through manipulation of the concentration of formamide within the range of 0% to 50%.

In practicing the present invention, amplification of either the nucleic acid probe or a sodA gene from the microorganism sample may be performed prior to the hybridization. Examples of amplification techniques include Strand Displacement Amplification (i.e., SDA), the Polymerase Chain Reaction (i.e., PCR), Reverse Transcription Polymerase Chain Reaction (i.e., RT-PCR), Nucleic Acid Sequence-Based Amplification (i.e., NASBA), Self-Sustained Sequence Replication (i.e., 3SR), and the Ligase Chain Reaction (i.e., LCR). (see, e.g. Innis et al., PCR Protocols, a Guide to Methods and Applications, eds., Academic Press (1990)).

The primers used to amplify the sample nucleic acids are oligonucleotides of defined sequence selected to hybridize selectively with particular portions of the sodA gene, in particular those that amplify the sodA internal fragment (sodAᵢₙₜ). A primer or primer pair may be coupled to a detectable moiety. A preferred example of such a detectable moiety is fluorescein, which is a standard label used in nucleic acid sequencing systems using laser light as a detection system. Other detectable labels can also be employed, however, including other fluorophores, radio-labels, chemical couplers such as biotin which can be detected with streptavidin-linked enzymes, and epitope tags such as digoxigenin detected using antibodies.

The present invention concerns methods for identification of species by a method which comprises providing a sample suspected of containing a gram positive bacteria, hybridizing a specific probe for a sodA gene or fragment thereof to nucleic acids from the microorganism, and detecting the presence or absence of hybridization. More specifically, the present invention concerns a method for the identification of a gram positive bacterial species selected from the group consisting of Streptococci, Staphlococci, Abiotrophia, and Enterococci, wherein the method has the steps of selecting a polynucleotide of 400 to 500 bp comprised between two conserved domains of SOD gene said polynucleotide having flanking regions consisting in two oligonucleotidic sequences and being specific for the genus or the species to be detected; hybridizing the DNA of the sample with the polynucleotide; washing the hybridized sample; visualizing the reaction of hybridization with an electric or electronic or calorimetric system. A polynucleotide of about 425 to 445 bp is particularly preferred.

The present invention also includes diagnostic kits for performing the analysis. These kits can be used to facilitate detection and identification of specific bacterial species in a clinical laboratories. Such kits would include instruction cards and vials containing the various solutions necessary to conduct a nucleic acid hybridization assay. These solutions would include lysing solutions, hybridization solutions, combination lysing and hybridization solutions, and wash solutions. The kits would also include labeled probes. The UP9A probe could be either unlabeled or labeled depending on the assay format. Standard references for comparison of results would also be necessary to provide an easy estimate of bacterial numbers in a given solution. Depending upon the label used additional components may be needed for the kit, e.g. enzyme labels require substrates.

Having generally described this invention, a further understanding can be obtained by reference to certain specific examples which are provided herein for purposes of illustration only, and are not intended to be limiting unless otherwise specified.

### EXAMPLES

The main characteristics of the bacterial strains used in this study, including the type strains, are listed in Table 1 and 2. Rapid extraction of bacterial genomic DNA was carried out by using the InstaGene™ Matrix (Bio-Rad, Hercules, Calif.) on cells collected from 2 ml of an overnight culture. The soda degenerate primers dl (5′-CCITAYICITAYGAYGCIYTIGARCC-3′) (SE Q ID NO:95) and d2 (5′-ARRTARTAIGCRTGYTCCCAIACRTC-3′) (SEQ ID NO:96) were used to amplify an internal fragment designated sodaAᵢₙₜ representing approximately 85% of their soda genes. PCRs were performed on a Gene Amp System 9600 instrument (Perkin Elmer Cetus, Roissy, France) in a final volume of 50 μl containing 250 ng of DNA as template, 0.5 μM of each primer, 200 μM of each dNTP, and 1 U of AmpliTaq Gold DNA polymerase (Perkin Elmer) in a 1X amplification buffer (10 mM Tris-HCI[pH 8.3), 50 mM KCl, 1.5 mM MgCl₂). The PCR mixtures were denatured (3 min at 95° C.), then subjected to 30 cycles of amplification (60 s of annealing at 37° C., 60 s of elongation at 72° C., and 30 s of denaturation at 95° C.), and 72° C. for 7 min for the last elongation cycle. A single DNA fragment corresponding to the expected 480-bp amplification product, sodAᵢₙₜ, was observed in all cases following agarose gel electrophoresis and ethidium bromide staining (data not shown). PCR products were purified on a S-400 Sephadex column (Pharmacia, Uppsala, Sweden) and directly sequenced on both strands with the oligos d1 and d2 by using the ABI-PRISM(O big dye terminator sequencing kit on a Genetic ABI-PRISM® 310 Sequencer Analyzer (Perkin Elmer). The cycle sequencing protocol was optimized as follows: the sequencing mixtures were subjected to 40 cycles of amplification consisting of 10 s of denaturation at 96° C., 5 s of annealing at 40° C., and 4 min of elongation at 60° C.

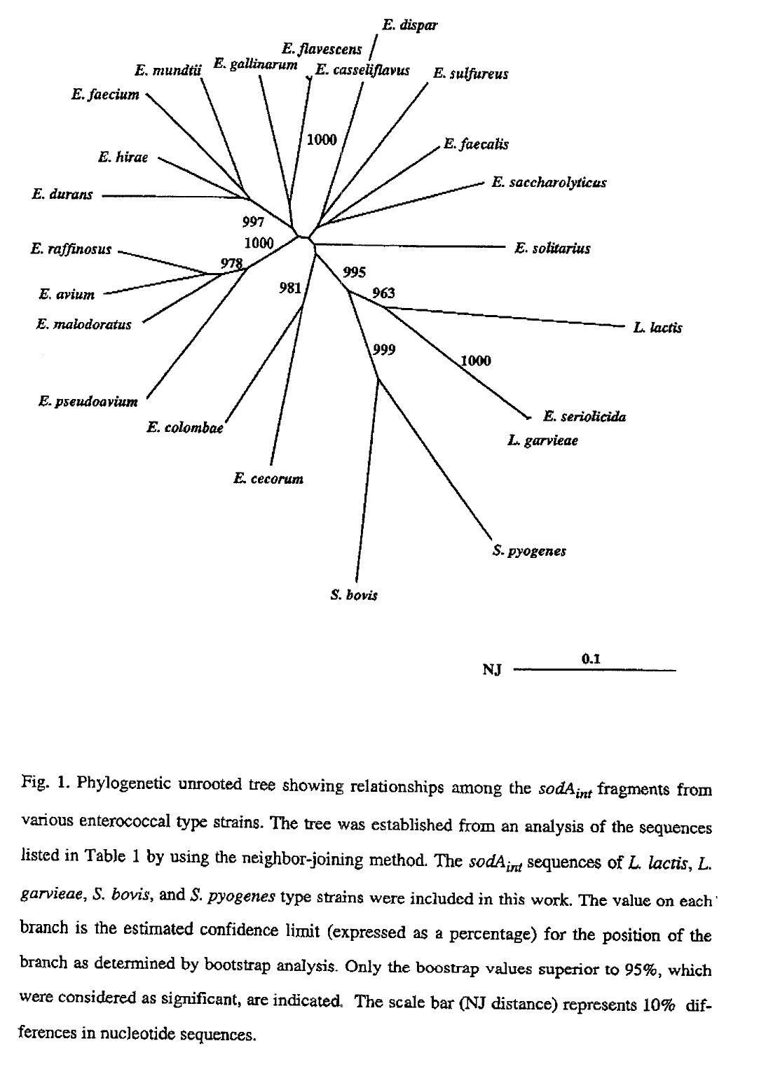

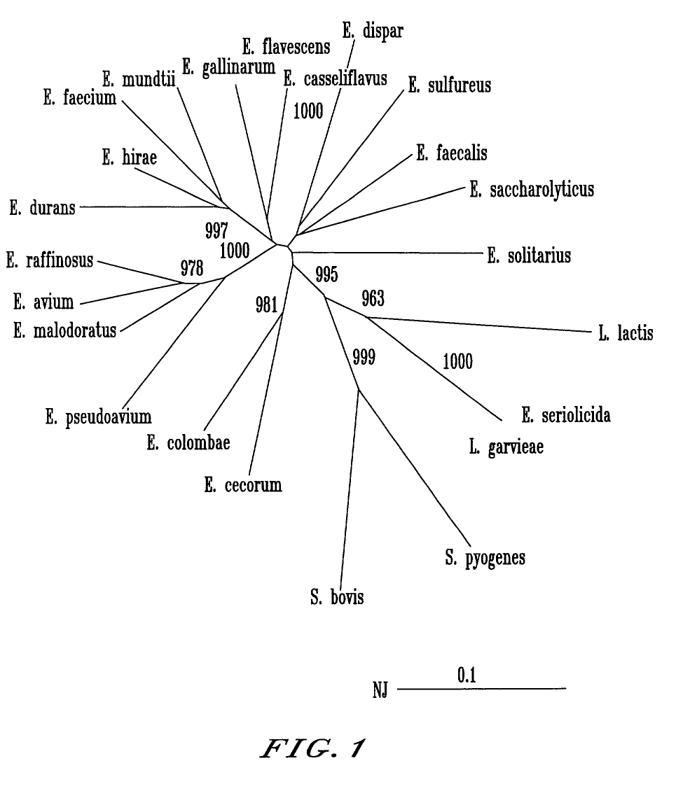

The nucleotide sequences of the sodAᵢₙₜ fragments from the type strains of E. avium, E. casseliflavus, E. cecorum, E. columbae, E. dispar, E. durans, E. faecalis, E. faecium, E. flavescens, E. gallinarum, E. hirae, E. malodoratus, E. mundtii, E. pseudoavium, E. raffinosus, E. saccharolyticus, E. seriolicida, E. solitarius, E. sulfureus, and Lactococcus garvieae were determined (Table 1). We assumed that the PCR products sequenced were actual sodaᵢₙₜ fragments since the corresponding deduced polypeptides all contained the amino acids characteristic of the manganese-dependent superoxide dismutase (16, 17) at the expected positions (data not shown). Multiple alignment of these sodAᵢₙₜ DNA sequences plus those from L. garvieae (Table 1), Lactococcus lactis (19), Streptococcus bovis (20) and Streptococcus pyogenes (20) was carried out by the Clustal X program (12) and an unrooted phylogenetic tree was constructed by the neighbor-joining (NJ) method (21). The sequence of the degenerate oligonucleotides d1 and d2 and alignment gaps were not taken into consideration for calculations. The reliability of the tree nodes was evaluated by calculating the percentage of 1,000 bootstrap resamplings that support each topological element. Only the nodes having a bootstrap value greater than 95% are indicated in FIG. 1 since this critical value could be used to define the monophyly of a lade of related organisms (7). This analysis revealed that, as expected, the members of the genus Enterococcus, with the exception of E. seriolicida were clustered within a clade supported by 99.5% of the bootstrap replicates. The sodAᵢₙₜ in sequences of E. seriolicida and of L. garvieae were almost identical (99.5% of sequence identity) and were clustered with that of L. lactis within a clade supported by 96.3% of the bootstrap confidence (Table 3 and FIG. 1). These results are consistent with the redesignation of E. seriolicida as L. garvieae (4). The phylogenetic tree representing the enterococcal sodAᵢₙₜ sequences (FIG. 1) has the same topology as the NJ tree constructed from the analysis of their 16S rDNA sequences (18). It is worth noting that the sodAᵢₙₜ sequences of E. casseliflavus and E. gallinarum type strains displayed 16.9% of sequence divergence, a value similar to the 19.7% of sequence divergence observed between the ddl genes encoding the D-Ala-D-Ala ligases in these species (5). These results do not support the suggestion that E. casseliflavus and E. gallinarum comprise a single species (18). By contrast, the fact that the 16S rDNA (18), the ddl (15), the vanC (3), and the sodAᵢₙₜ (Table 3) genes of E. casseliflavus and E. flavescens type strains were almost identical (99.9, 99.5%, 96%, and 98% of sequence identity, respectively) suggest that they should be associated in a single species.

The phylogenetic tree showed in FIG. 1 revealed the presence of two major clusters within the enterococcal species which we have designated the faecium group (E. faecium, E. durans, E. hirae, and E. mundtii) and the avium group (E. avium, E. malodoratus, E. pseudoavium, and E. raffinosus). Within each group, the 16S rDNA sequences exhibited more than 99% of sequence identity (18) whereas the highest percentage of similarity found between two sodAᵢₙₜ sequences was 87.9% (Table 3). These results confirm that the gene sodA constitutes a more discriminative target sequence than the 16S RNA to differentiate closely related bacterial species.

Fifteen enterococcal isolates were identified by using conventional microbiological tests, ID 32 Strep, and the sodAᵢₙₜ systems (Table 2). In all cases, the sodAᵢₙₜ sequences of the isolates displayed less than 1.5% of divergence with that of the corresponding type strain. For ten strains (NEM1616, NEM1617, NEM1621, NEM1623, NEM1624, NEM1625, NEM1626, NEM 1627, NEM1628, and NEM 1630), the two methods gave the same results. Four isolates (NEM1618, NEM1620, NEM1622, AND NEM1629) were identified at the species level with the sodAᵢₙₜ system but not with the conventional microbiological tests and the ID 32 Strep system. The remaining isolate NEM1619 was identified with the ID 32 Strep system as E. hirae but was identified with the sodAᵢₙₜ system as E. durans (Table 2). The reliability of the molecular identification of NEM1164 was based on the fact that its sodAᵢₙₜ fragment displays 99.5% and 85% of sequence identity with those of the type strains of E. durans and E. hirae, respectively.

In conclusion, we have determined the sodAᵢₙₜ sequences of the type strains of E. avium, E. casseliflavus/E. flavescens, E. cecorum, E. columbae, E. dispar, E. durans, E. faecalis, E. faecium, E. gallinarum, E. hirae, E. malodoratus, E mundtii, E. pseudoavium, E. raffinosus, E. saccharolyticus, E. seriolicida, E. solitarius, and E. sulfureus and demonstrated the usefulness of this database for the species identification of enterococcal isolates. The identification method presented in this study is not accessible to routine clinical microbiology laboratories but it may become the gold-standard technique in reference and large research hospital laboratories for epidemiologic purposes and/or to identify problematic strains.

Other polynucleotide sequences specific for species of Staphlococci, Streptococci and Abiotrophia have also been identified by using the same method. These sequences correspond to SEQ ID NOS:54-59, SEQ ID NOS:37-58 and SEQ ID NOS:51-53, respectively.

Obviously, numerous modifications and variations on the present invention are possible in light of the above teachings. It is therefore to be understood that within the scope of the appended claims, the invention may be practiced otherwise than as specifically described herein.

### REFERENCES

1. Cartwright, C. P., F. Stock, G. A. Fable, and V. J. Gill. 1995. Comparison of pigment production and motility tests with PCR for reliable identification of intrinsically vancomycin-resistant enterococci. J. Clin. Microbiol. 33:1931-1933.

2. Devriese, L. A., B. Pot, K. Kersters, S. Lauwers, and F. Haesebrouck. 1996. Acidification of methyl-alpha-D-glucopyranoside: a useful test to differentiate Enterococcus casseliflavus and Enterococcus gallinarum from Enterococcus faecium species group and from Enterococcus faecalis. J. Clin. Microbiol. 34:2607-2608.

3. Dutka-Malen, S., S. Evers, and P. Courvalin. 1995. Detection of glycopeptide resistance genotypes and identification to the species level of clinically relevant enterococci by PCR. J. Clin. Microbiol. 33:1434.

4. Eldar, A., C. Ghittino, L. Asanta, E. Bozzetta, M. Goria, M. Prearo, and H.

Bercovier. 1996. Enterococcus seriolicida is a junior synonym of Lactococcus garvieae, a causative agent of septicemia and meningoencephalitis in fish. Curr.

Microbiol. 32:85-88.

5. Evers, S., B. Casadewall, M. Charles, S. Dutka-Malen, M. Galimand, and P.

Courvalin. 1996. Evolution of structure and substrate specificity in D-alanine:D-alanine ligases and related enzymes. J. Mol. Evol. 42:706-712.

6. Facklam, R. R., and M. D. Collins. 1989. Identification of Enterococcus species isolated from human infections by a conventional test scheme. J. Clin. Microbiol. 27:731-734.

7. Felsenstein, J. 1985. Confidence limits on phylogeny and approach using the boostrap. Evolution 39:783-791,

8. Fox, G. E., J. D. Wisotzkey, and P. Jurtshuk, Jr. 1992. How close is close: 16S rRNA sequence identity may not be sufficient to guarantee species identity. Int. J. Syst. Bacteriol. 42:166-170.

9. Hunt, C. P. 1998. The emergence of enterococci as a cause of nosocomial infection.

Br. J. Biomed. Sci. 55:149-156.

10. Huycke, M. M., D. F. Sahm, and M. 3. Gilmore. 1998. Multiple-drug resistant enterococci: the nature of the problem and an agenda for the future. Emerg. Infect. Dis. 4:239-249.

11. Iwen, P. C., D. M. Kelly, J. Linder, and S. H. Hinrichs. 1996. Revised approach for identification and detection of ampicillin and vancomycin resistance in Enterococcus species by using MicroScan panels. J. Clin. Microbiol. 34:1779-1783.

12. Jeanmougin, F., J. D. Thompson, M. Gouy, D. G. Higgins, and T. J. Gibson. 1998. Multiple sequence alignment with Clustal X. Trends Biochem. Sci. 23:403-405.

13. Moellering, R. C., Jr. 1998. Vancomycin-resistant enterococci. Clin. Infect. Dis. 26:1196-1199.

14. Monstein, H. J., M. Quednau, A. Samuelsson, S. Ahrne, B. Isaksson, and J. Jonasson. 1998. Division of the genus Enterococcus into species groups using PCR-based molecular typing methods. Microbiology 144:1171-1179.

15. Navarro, F., and P. Courvalin. 1994. Analysis of genes encoding D-alanine-D-alanine ligase-related enzymes in Enterococcus casseliflavus and Enterococcus flavescens. Antimicrob. Agents Chemother. 38:1788-1793.

16. Parker, M. W., and C. C. F. Balke. 1988. Crystal structure of manganese superoxide dismutase from Bacillus stearothermophilus at 2.4 A resolution, J. Mol. Biol. 199:649-661.

17. Parker, M. W., and C. C. F. Blake. 1988. Iron- and manganese-containing superoxide dismutases can be distinguished by analysis of their primary structures. FEBS Lett. 229:377-382.

18. Patel, R., K. E. Piper, M. S. Rouse, J. M. Steckelberg, J. R. Uhl, P. Kohnerg M. K. Hopkins, F. R. Cockerill, 3rd, and B. C. Kline. 1998. Determination of 16S rRNA sequences of enterococci and application to species identification of nonmotile Enterococcus gallinarum isolates. J. Clin. Microbiol. 36:3399-3407.

19. Poyart, C., P. Berche, and P. Trieu-Cuot. 1995. Characterization of superoxide dismutase genes from Gram-positive bacteria by polymerase chain reaction using degenerate primers, FEMS Microbiol. Lett. 131:41-45.

20. Poyart, C., G. Quesne, S. Coulon, P. Berche, and P. Trieu-Cuot. 1998. Identification of streptococci to species level by sequencing the gene encoding the manganese-dependent superoxide dismutase. J. Clin. Microbiol. 36:41-47.

21. Saitou, N., and M. Nei. 1987. The neighbor-joining method: a new method for reconstructing phylogenetic trees. Mol. Biol. Evol. 4:406-425.

22. Tyrrell, G. J., R. N. Bethune, B. Willey, and D. E. Low. 1997. Species identification of enterococci via intergenic ribosomal PCR. J. Clin. Microbiol. 35:1054-1060.

23. Ueda, K., T. Seki, T. Kudo, T. Yoshida, and M. Kataoka. 1999. Two distinct mechanisms cause heterogeneity of 16S rRNA. J. Bacteriol. 181:78-82.

24. Woodford, N. 1998. Glycopeptide-resistant enterococci: a decade of experience. J. Med. Microbiol. 47:849-862.

1. A method for accurate identification of the species of a gram positive bacteria in a sample comprising
providing a sample suspected of containing said gram positive bacteria;
hybridizing a specific probe for a sodA gene or a fragment thereof to nucleic acids from said microorganism; and
detecting the presence or absence of hybridization.

2. The method according to claim 1, further comprising amplification of said sodA gene from the microorganism prior to said hybridizing.

3. The method according to claim 1, wherein said microorganism is selected from the group consisting of Enterococci, Abiotrophia, Streptococci and Staphylococci.

4. The method according to claim 3, wherein said microorganism is an Enterococci and is selected from the group consisting of E. avium, E. casseliflavus, E. cecorum, E. columbae, E. dispar, E. durans, E. faecalis, E. faecium, E. flavescens, E. gallinarum, E. hirae, E. malodoratus, E. mundtii, E. pseudoavium, E. raffinosus, E. saccharolyticus, E. seriolicida, E. solitarius, and E. sulfureus

5. The method of claim 1, wherein specific probe is selected from the group consisting of SEQ ID NOS:1-94.

6. The method according to claim 3, wherein said microorganisms is a Staphyloccus and is selected from the group consisting of S. arlettae, S. auricularis, S. capitis subspecies capitis, S. capitis subspecies ureolyticus, S. caprae, S. carnosus subspecies carnosus, S. carnosus subspecies utilis, S. chromogenes, S cohnii subspecies cohnii, S. cohnii subspecies urealyticum, S. condimenti, S. delphini, S epidermidis, S. equorum, S felis, S. gallinarum, S. haemolyticus, S. hominis subspecies hominis, S. hominis subspecies novobiosepticus, S. hyicus, S. intermedius, S. kloosii, S. lentus, S. lugdunensis, S. luntae, S. muscae, S. pasterui, S. piscifermentans, S. pulvereri, S. saccharolyticus, S. saprophyticus subspecies bovis, S. saprophyticus subspecies saprophyticus, S schleiferi subspecies coagulans, S. schleiferi subspecies schleiferi, S. sciuri subspecies carnaticus, S. sciuri subspecies sciuri, S. simulans, S vitulinus, S. warneri, and S. xylosus.

7. A polynucleotide or fragment thereof specifically hybridizing to an Enterococcus microorganism, wherein SEQ ID NO:1 is specific for E. avium, SEQ ID NO:2 is specific for E. casseliflavus, SEQ ID NO:3 is specific for E. cecorum, SEQ ID NO:4 is specific for E. columbae, SEQ ID NO:5 is specific for E. dispar, SEQ ID NO:6 is specific for E. durans, SEQ ID NO:7 is specific for E.faecalis, SEQ ID NO:8 is specific for E. faecium, SEQ ID NO:9 is specific for E. flavescens, SEQ ID NO:10 is specific for E. gallinarum, SEQ ID NO:11 is specific for E. hirae, SEQ ID NO:12 is specific for E. malodoratus, SEQ ID NO:13 is specific for E. mundtii, SEQ ID NO:14 is specific for E. pseudoavium, SEQ ID NO:17 is specific for E. raffinosus, SEQ ID NO:15 is specific for E. saccharolyticus, SEQ ID NO:18 is specific for E. seriolicida, SEQ ID NO:16 is specific for E. solitarius, and SEQ ID NO:19 is specific for E. sulfureus

8. A polynucleotide or fragment thereof specifically hybridizing to a microorganism of the genus Enterococci, wherein SEQ ID NOS:21-36 are specific for species in the Enterococci.

9. A polynucleotide or fragment thereof specifically hybridizing to a microorganism of the genus Lactococcus garvieae, wherein said polynucleotide is SEQ ID NO:20.

10. A polynucleotide or fragment thereof specifically hybridizing to a microorganism of the genus Streptococcus, wherein SEQ ID NOS:37-50 are specific for species in the Streptococci.

11. A polynucleotide or fragment thereof specifically hybridizing to a microorganism of the genus Abiotrophia, wherein SEQ ID NOS:51-53 are specific for species in the Abiotrophia.

12. A polynucleotide or fragment thereof specifically hybridizing to a microorganism of the genus Staphlococcus, wherein SEQ ID NOS:54-93 are specific for species in the Staphlococcus.

13. A DNA chip comprising at least one polynucleotide or a fragment thereof according to claims 7, 8, 9, 10, 11, or 12.

14. The method according to claim 1, wherein said fragment of sodA is sodAᵢₙₜ.

15. A method for the identification of a gram positive bacterial species selected from the group consisting of Streptococci, Staphylococci, Abiotrophia and Enterococci comprising
selecting a polynucleotide of about 425 to 445 bp comprised between two conserved domains of SOD gene said polynucleotide having flanking regions consisting in two oligonucleotidic sequences and being specific for the genus or the species to be detected;
hybridizing the DNA of the sample with the polynucleotide;
washing the hybridized sample;
visualizing the reaction of hybridization with an electric or electronic or calorimetric system.

16. A kit for the detection of a gram positive bacteria present in a sample containing at least a polynucleotide in SEQ ID NOS: 1-94.

17. A 400 bp polynucleotide sequence obtained after amplification of a DNA template from a sample by using a pair of primers SEQ ID NOS:95 and 96, wherein said pair of primers is specific for the SOD gene of a gram positive bacteria.

18. The method of claim 15, wherein the polynucleotide is about 429 bp and is specific for a Staphylococi species.

19. The method of claim 15, wherein the polynucleotide is about 435 and is specific for Streptococci species.

20. The method of claim 15, wherein the polynucleotide is about 438 bp and is specific for Enterococci species.

21. The method of claim 15, wherein the polynucleotide is about 438 to 441 bp and is specific for Abiotrophia species.
TABLE 1
Enterococcal type strains used in this study

| | Other | sodAᵢₙₜ |
|---|---|---|
| Strain^{a} | designation^{b} | accession n° |
| E. avium CIP 103019 T | ATCC 14025 | AJ387906 |
| E. casseliflavus CIP 103018 T | ATCC 25788 | AJ387907 |
| E. cecorum CIP 103676 T | ATCC 43198 | AJ387908 |
| E. columbae CIP 103675 T | ATCC 51263 | AJ387909 |
| E. dispar CIP 103646 T | ATCC 51266 | AJ387910 |
| E. durans CIP 55.125 T | ATCC 19432 | AJ387911 |
| E. faecalis CIP 103015 T | ATCC 19433 | AJ387912 |
| E. faecium CIP 103014 T | ATCC 19434 | AJ387913 |
| E. flavescens CIP 103525 T | ATCC 49996 | AJ387914 |
| E. gallinarum CIP 103013 T | ATCC 49573 | AJ387915 |
| E. hirae CIP 53.48 T | ATCC 8043 | AJ387916 |
| E. malodoratus CIP 103012 T | ATCC 43197 | AJ387917 |
| E. mundtii CIP 103010 T | ATCC 43186 | AJ387918 |
| E. pseudoavium CIP 103647 T | ATCC 49372 | AJ387919 |
| E. saccharolyticus CIP 103246 T | ATCC 43076 | AJ387920 |
| E. solitarius CIP 103330 T | NCTC 12193 | AJ387921 |
| E. raffinosus CIP 103329 T | ATCC 49427 | AJ387922 |
| E. seriolicida CIP 104369 T | ATCC 49156 | AJ387923 |
| E. sulfureus CIP 1043737 T | DSM 6905 | AJ387924 |
| L. garvieae CIP 102507 T | DSM 20684 | AJ387925 |

^{a}CIP, Collection de l'Institut Pasteur.
^{b}ATCC, American Type Culture Collection; DSM, Deutsche Sammlung Von Mikrooganismen; NCTC, National Collection of Type Cultures.
TABLE 2
Identification of various enterococcal strains by sequencing the sodAᵢₙₜfragment.

| Strain | Relevant characteristics^{a} | Bacterial species^{b} | Accession number |
|---|---|---|---|
| NEM1616 | E. faecalis; vanA | E. faecalis (99.5) | AJ387927 |
| NEM1617 | E. faecalis; vanA | E. faecalis (98.6) | AJ387928 |
| NEM1618 | Enterococcus sp. | E. durans (99.3) | AJ387929 |
| NEM1619 | E. hirae | E. durans (99.5) | AJ387930 |
| NEM1620 | Enterococcus sp. | E. durans (99.1) | AJ387931 |
| NEM1621 | E. hirae | E. hirae (99.8) | AJ387932 |
| NEM1622 | Enterococcus sp. | E. hirae (99.5) | AJ387933 |
| NEM1623 | E. casseliflavus | E. casseliflavus (99.1) | AJ387934 |
| NEM1624 | E. faecium; vanB | E. faecium (99.5) | AJ387935 |
| NEM1625 | E. faecium; vanA | E. faecium (100) | AJ387936 |
| NEM1626 | E. faecium; vanB | E. faecium (99.8) | AJ387937 |
| NEM1627 | E. faecium; multiply resistant strain | E. faecium (99.8) | AJ387938 |
| NEM1628 | E. faecium; multiply resistant strain | E. faecium (99.8) | AJ387939 |
| NEM1629 | Enterococcus sp. | E. gallinarum (98.6) | AJ387940 |
| NEM1630 | E. avium | E. avium (100) | AJ387941 |

^{a}Bacterial strains were all clinical isolates from our collection which were identified by using conventional microbiological tests and the ID 32 Strep System (API-bio-Merieux). Presence of vanA (NEM1616, NEM1617, and NEM1625) and vanB (NEM1624 and NEM1626) was determined by PCR with specific primers (3).
^{b}The species identification was based on the phylogenic position of the sodAᵢₙₜ fragment of the strain studied relative to those of the type strains, as shown in FIG. 1. The number in parentheses indicates the percentage of identity of the sodAᵢₙₜ fragment with that of the corresponding type strains.
TABLE 3
Identity matrix based on pairwise comparisons of sodAᵢₙₜ fragments of enterococcal type strains

| | % of identity with: | | | | | | | | | | | | | | | | | |
|---|---|---|---|---|---|---|---|---|---|---|---|---|---|---|---|---|---|---|
| Strain^{a} | 2 | 3 | 4 | 5 | 6 | 7 | 8 | 9 | 10 | 11 | 12 | 13 | 14 | 15 | 16 | 17 | 18 | 19 |
| 1 C. avium | 74.0 | 67.4 | 71.9 | 70.3 | 70.3 | 73.7 | 69.9 | 74.2 | 75.1 | 75.6 | 71.0 | 80.1 | 72.6 | 74.0 | 85.4 | 87.9 | 60.7 | 67.1 |
| 2 E. casseliflavus | | 66.4 | 70.8 | 72.6 | 72.4 | 77.9 | 72.4 | 99.5 | 83.1 | 78.5 | 77.4 | 71.5 | 73.5 | 74.0 | 76.7 | 76.7 | 66.4 | 75.6 |
| 3 E. cecorum | | | 78.8 | 72.4 | 66.0 | 68.7 | 66.2 | 66.9 | 67.4 | 70.3 | 64.2 | 65.5 | 71.7 | 65.8 | 67.6 | 66.0 | 62.8 | 68.7 |
| 4 E. colombae | | | | 69.4 | 68.9 | 71.7 | 69.2 | 70.8 | 72.6 | 73.1 | 68.7 | 69.6 | 72.1 | 72.8 | 73.3 | 71.9 | 67.1 | 69.2 |
| 5 E. dispar | | | | | 70.3 | 77.4 | 68.7 | 72.8 | 72.1 | 73.5 | 72.8 | 70.5 | 71.0 | 69.4 | 72.1 | 70.3 | 62.6 | 74.9 |
| 6 E. durans | | | | | | 73.1 | 81.3 | 72.4 | 76.3 | 84.9 | 80.1 | 69.6 | 72.8 | 70.5 | 71.5 | 73.3 | 62.1 | 73.7 |
| 7 E. faecalis | | | | | | | 72.6 | 78.3 | 77.6 | 77.9 | 72.4 | 71.2 | 78.8 | 73.5 | 77.9 | 75.1 | 67.1 | 76.5 |
| 8 E. faecium | | | | | | | | 72.4 | 77.2 | 83.1 | 81.7 | 67.4 | 72.6 | 69.4 | 71.9 | 71.9 | 62.1 | 72.1 |
| 9 E. flavescens | | | | | | | | | 83.1 | 78.3 | 77.2 | 72.1 | 72.8 | 74.4 | 77.2 | 76.9 | 65.8 | 74.9 |
| 10 E. gallinarum | | | | | | | | | | 80.8 | 78.5 | 73.3 | 76.0 | 73.5 | 77.4 | 75.1 | 66.2 | 76.7 |
| 11 E. hirae | | | | | | | | | | | 83.6 | 71.5 | 73.3 | 73.5 | 77.4 | 76.7 | 63.5 | 75.3 |
| 12 E. mundtii | | | | | | | | | | | | 70.8 | 69.4 | 69.6 | 71.9 | 73.5 | 63.9 | 74.4 |
| 13 E. pseudoavium | | | | | | | | | | | | | 70.5 | 69.2 | 81.7 | 80.4 | 62.8 | 65.3 |
| 14 E. saccharolyti- | | | | | | | | | | | | | | 72.4 | 75.1 | 71.0 | 62.1 | 76.7 |
| cus | | | | | | | | | | | | | | | | | | |
| 15 E. solitarius | | | | | | | | | | | | | | | 74.2 | 72.8 | 64.8 | 72.1 |
| 16 E. malodoratus | | | | | | | | | | | | | | | | 87.9 | 63.5 | 70.5 |
| 17 E. raffinosus | | | | | | | | | | | | | | | | | 64.6 | 67.6 |
| 18 E. seriolicida | | | | | | | | | | | | | | | | | | 61.6 |
| 19 E. sulfureus | | | | | | | | | | | | | | | | | | — |

^{a}The main characteristics of the strains are listed in table 1.


SEQUENCE LISTING (96 sequences)

SEQ ID NO: 1 - DNA, 438 bp, Enterococcus avium
tatatcgatgttgaaacgatgcatttgcatcatgacaaacaccataacacttatgtaacaaatttaaatgctgcgattgaaaaatatccggaattagaagaacagtcaattgaagagctaatgaaaaacttaaatgaagttcctgaggacattcgtacggctgtacgtaataacggcggcggacatgctaaccacagcttcttctggaaaattatggctccaaatgctggtggtgaacctacaggcgcgattaaggacgcaattgatcaagcatttggcagctttgaaaaaatgaaggaagaattcaagactgcagcaactggtcgttttggttctggctgggcatggttagtattgaacaatggaaaattagaaattacttctactgcaaatcaagacagcccattaactgatggaaaaacaccgatcattggctta

SEQ ID NO: 2 - DNA, 438 bp, Enterococcus casseliflavus
tatattgatgaagaaacgatgcatttgcatcatgataaacaccacaacacttatgtaacaaacttaaatgcagcgattgaaaaacatcctgaattaggtgaaaaaacagttgaagaattattagcagacttttcttctgtacctgaagatattcaaacagcggttcgcaacaatggcggcggccatgctaaccacacgttcttctgggaaatcttaggcccaaatgctggtggcgaacctactggggcaatcaaagaggcaattgaagaaacattcggcagctttgaagactttaaagaagaatttaaaactgctgcaactggacgttttggttcaggttgggcatggttagtcgttaaagacggtaaactagcagtcacttcaacagcgaatcaagattcaccattgatggatggtcaaacacctgttttaggttta

SEQ ID NO: 3 - DNA, 438 bp, Enterococcus cecorum
acaatcgatgaagaaacaatgcatctacatcatgaaaaacatcataaaacctatttaacaaatttaaatgcggctttagaaaaacatccagagttgccagaaaaatctattgaagacttattagctggtatcaatgaagtgcctgctgatattcgccaagctgttattaataatggtggtggacacgcaaaccattcattcttctggaaaattatgacgccaaacggtcaaggtgcgcctgtgggtgaattaaaagctgctattgacgaaacttttggtagcttcgatgaattcaaggcacaatttaaagctgctgcggctagtcgttttggttcaggttgggcttggttagttgtcgacaatggtaaattagctattatttctactgcgaaccaagattcaccattaatggaaggcaaaacaccagttgttgggctt

SEQ ID NO: 4 - DNA, 438 bp, Enterococcus columbae
acaatcgatgaagaaacaatgcatctacatcatgaaaaacatcacaacacttacgttactaatttaaatgctgcaattgaaaaacatccagaatttggtaccaagacagttgaagaattagtggctgcaattaatgaagtgcctgaagatattcgtacggctgtccgtaacaatggtggtggtcatgcgaaccattcattcttctggaaaattatgtctccaaatggtggcggtgaaccagttggtgaattaaaagctgccattgaagaagcttttggtagctttgatgaatttaaggctcaatttaaagcagcagcagcagctcgctttggctctggctgggcatggttagtagtcgataacggtaaattagcaattatttcaacagcaaaccaagataatccattaatggaaggtaaagtacctgtcgttggctta

SEQ ID NO: 5 - DNA, 438 bp, Enterococcus dispar
tatatcgacgtggagacaatgcacttacaccacgataaacatcacaacacatatgtaacaaatttaaacgctgctttggaaaaatatcctgaactagcagaaaaaagtgtggaagaattaattgcctatatggatgaaattcctgctgatattcgtactgctgttcaaaataatggtggtggacatgcaaaccatacattcttttgggaaattatggcaccaaatgctggtggaacgccaactggagctttaaaggatgctattgacgaaacatttggttcttttgaagatttcaaaagtgaatttaaaactgctgcgacaggacgtttcggttctggttgggcatggttagtggtaaataacggtaaattatctatcatgtcaactgcgaaccaagattcaccattaatggaaggcaaaactcccattatcggttta

SEQ ID NO: 6 - DNA, 438 bp, Enterococcus durans
tatatcgatgaagaaacgatgcacttgcatcatgacaaacaccataatacttatgttacaaatttaaacgcagctattgaaaagtatccagaattaggcgaaaaatcagtggaagaattgctttctgatatggacgcgattcctactgatattaagacagcggtacaaaacaatggcggtggacatgcaaaccattcatttttctggaaaatcatggcacctaatgcaggtggcgaaccaacaggcgaaatcaaagaagcgattgatgaagcttttggtgatttcgcaacattcaaagaagagttcaagaaagcggctgccggacgctttggatcaggttgggcatggttagtattggaagatggtaaattggcaatcacttctacagcaaaccaagattctccattgatgacaggccaaacacctatcttaggatta

SEQ ID NO: 7 - DNA, 438 bp, Enterococcus faecalis
tacattgacgtggaaacaatgcacttacaccatgataaacaccacaacacttatgtgactaacttaaacgcagcgattgaaaaacatccagaattaggcgaaaaatctgtagaagacctaatttcagatatgaatgctattcctgaagatatccgtacagccgttcgtaacaatggtggcggtcacgcaaaccaaacattcttctgggaaattatggcaccaaatgctggtggacaaccaactggcgctattaaagaagcaatcgatgaaacatttggtagctttgatgaaatgaaagctgctttcaaaacagctgcaactggccgctttggttcaggttgggcttggttagttgtgaataacggtaaattagaaatcacttcaacaccaaaccaagattcaccattaatggatggccaaacacctgttttaggtctt

SEQ ID NO: 8 - DNA, 438 bp, Enterococcus faecium
tatattgacgaagaaacgatgcatctgcatcatgataagcatcacaatacttatgtgacgaatttaaattcagcaattgagaaatacccagaattaggcgaaaaaacaatagaagaattattatctgatatggacgctattccaacagatatcaagacagctgtacgtaacaatggtggcggacatgctaaccattcatttttctgggaaatcatggcaccaaatgctggtggcgaacctacaggagaaataaaagaagcgattaatgaagcttttggtgatttttcttcttttaaagaagaattcaaaaaagcagccgctggacgatttggttctggatgggcttggcttgtaatggaaaatggaaaattagctattacctctactgcaaatcaagattctccattgatggaaggaaagacaccaattctaggtttg

SEQ ID NO: 9 - DNA, 438 bp, Enterococcus flavescens
tatattgatgaagaaacgatgcatttgcatcatgataaacaccacaacacttatgtaacaaacttaaatgcagcgattgaaaaacatcctgaattaggtgaaaaaaaagttgaagaattattagcagacttttcttctgtacctgaagatattcaaacagcggttcgcaacaatggcggcggccatgctaaccacacgttcttctgggaaatcttaggcccaaatgctggtggcgaacctactggggcaatcaaagaggcaattgaagaaacattcggcagctttgaagactttaaagaagaatttaaaactgctgcaactggacgttttggttcaggttgggcatggttagtcgttaaagacggtaaactagcaatcacttcaacagcgaatcaagattcaccattgatggatggtcaaacacctgttttaggttta

SEQ ID NO: 10 - DNA, 438 bp, Enterococcus gallinarum
tacattgatgaagaaacgatgcatttgcatcatgacaagcatcacaatacttacgtcacaaatttgaatgcagcaattgaaaaacatcctgaattaggtgaaaaatcagttgaagaattacttgctgattttgattcggttcctgaagacatcaaaacagctgtccgtaataacggtggtggtcatgcaaatcacagctttttctgggaaatcttggcaccaaatgctggtggtgaaccaacaggagccatcaaagaagccatcgaagaaacatttggcagctttgctgatttcaaagaagaattcaaaacagcagcaactggccgctttggttctggctgggcttggttagtcatcaaagatggtaaattagcgatcacttccactgcgaaccaagattcaccattaatggatggtcaaacgccagttttaggttta

SEQ ID NO: 11 - DNA, 438 bp, Enterococcus hirae
tatatcgatgaagaaacgatgcacttgcatcatgacaaacaccataatacttatgtaacaaatttaaatgcagcgattgaaaaacatccagaactaggtgaaaaaacaatcgaagaactactttctgatatggatgctgtccctacagatatcaagactgctgtacgtaataatggtggcggacatgcaaaccattctttcttctggaaaatcatggcaccaaatgctggtggcgaaccaactggtgcaattaaagaagcgattgatgaagcctttggtgattttgcaacatttaaggaagaatttaaaaaagctgcagctggccgttttggttcaggttgggcttggttagtgatggaaaatggtaaattagcgatcacttcaacagccaatcaagattcaccattaatggaaggcaaaacacctattttaggttta

SEQ ID NO: 12 - DNA, 438 bp, Enterococcus malodoratus
tatatcgatgttgaaacgatgcatttgcatcatgacaagcaccataacacttatgtaaccaatttaaatgctgcgattgaaaaatatccagaattagcagaacaatcagtggaagaattagtaacgaacttgaatgaagtgccagaagatattcgtacggctgttcgcaacaatggcggaggtcatgcaaatcatagtttcttctggaaaatcatggcgccaaatgctggcggaaaaccaacaggtgcgatcaaagatgcaattgatgaagcattcggcagctttgaaaaaatgaaagaagaattcaaaacagctgcaactggccgctttggttctggctgggcttggctagtcttgaacaatggtaaattagaaattacttcaacaccaaatcaagataacccattaacagatggtaaaacaccaattattggttta

SEQ ID NO: 13 - DNA, 438 bp, Enterococcus mundtii
tatattgacgaagaaacgatgcatttgcatcatgacaaacatcacaatacttatgtgacaaacttaaatgcagcgatcgaaaaatatcctgaactaggtggaaaaacaatagaagaattggtttcagacatggatgctattccatctgacattcaaactgctgtacgtaataatggtggtggacatgcgaaccattcattcttctggaaaatcatggcaccaaatgctggtggcgaaccaacaggagcaatcaaagacgcaattaatgaaacattcggcgattttgcaacattcaaagaagaattcaaaaaagcagcagcaggacgtttcggttctggctgggcttggttagtacttgaagatggcaaacttgccatcacttctactgccaaccaagattcaccattgatggaaggcaagaaacctgttctaggttta

SEQ ID NO: 14 - DNA, 438 bp, Enterococcus pseudoavium
tacattgatgttgaaacgatgcacttgcatcatgataaacaccacaatacttatgttactaatttgaatgtagcaattgaaaaatatcctgaactagcggagcaatctgttgaggatttagttgcaaacttaaatgagttgcctgaagatattcagacggctgttcgtaacaatggcggtggtcatgcgaaccatagctttttctggaagatcatggcaccaaacgcgggtggtgcgccaactggtgcgatcaaagacgccattgacgaagctttcggcggctttgaaaaaatgaaagaagaattcaaacttgctgcgacaggacgttttggttctggttgggcttggttagtttggaacaatggcaagttggaaattacgtcaactgctaatcaagacaatccattgactgacgggaaaacaccaatcattggctta

SEQ ID NO: 15 - DNA, 438 bp, Enterococcus saccharolyticus
cacattgatgttgaaacaatgcatttacatcatgacaaacaccataacacttatgtgacaaacttaaatgcagcagttgaaaaatatcctgaattaggcgaaaaatctgtagaagatttaatttctgatttagcagcagttcctgaagatattcgcacagccgtacgcaacaatggtggtggacatgcaaaccatacattcttttgggaaattatggcaccaaacgctggtggcgaacctgtaggcgagctaaaagcagcgattgacgaaaaatttggtagctttgatgcattcaaagcagaatttaaagcagcagcgactagccgatttggttctggttgggcttggttagctttaaataatgggttattagaagtgacttctacaccaaatcaagattctccattaatggatggtcgtacaccaattgttggttta

SEQ ID NO: 16 - DNA, 438 bp, Enterococcus solitarius
tattttgacgaagagaccatgcatttgcatcatgataaacaccataacacttatgtgacgaacttaaatgcagcgattgaaaaacatcctgaattaggcgaaaaatcagtggaagacctaatggcagatcttgatagtgtccctgaagatatttttacagcagtacgtaataacggcggtggacatgtaaatcattctttcttctggaagattttatctccagatggaggcggtgaaccaaccggtgcattaaaagatgcgattgatcaagaatttggcagttttgatgcttttaaagatgaatttaaggcagctgccaccggtcgttttggttctggctgggcttggttagttttagataacggcaaattaaaaattacttcgacgccaaaccaagattctccattgacagatggacaaattcctattattggctta

SEQ ID NO: 17 - DNA, 438 bp, Enterococcus raffinosus
tatatcgatgttgaaacgatgcacttgcatcatgacaagcaccacaacacttatgtaaccaacttgaatgctgcgattgaaaaatatccagaattaggcgaacaatcaatcgaagaattagtgacgaacttgaatgaagttcctgaagacattcgtacagcggtacgtaataatggcggcggacatgcgaaccacagcttcttctggaaaatcatggcgcctaatgctggcggcgaaccaacaggtgcgatcaaagaagcaattgatcaagctttcggcagctttgagaaaatgaaggaagaattcaagacagcggcaacaggacgttttggttctggttgggcatggttggtattgaacaacggtaaattagaaattacatcaaccgcgaatcaagatagcccattgactgatggcaaaacaccaattattggttta

SEQ ID NO: 18 - DNA, 438 bp, Enterococcus seriolicida
ttctttgatgaagaaacaatgcacttgcaccatgacaaacatcaccaaacatacgtaaataatcttaatgcagcgattgaaaaacacccagaattctttgataaaactgttgaagaattagtggcttatttggaccgtttgccagaagacattcgtgttgcggtacgtaacaacggtggaggacacttgaaccacacaatgttctgggaatggctcgctccaaatgcaggtggtgcaccaacaggtgatatcgctgcagcaatcgatgaagcttttggttcatttgacgacttcaaagctgaatttaaagcagctgctacaggacgtttcggttcaggttgggcttggttagttcttgattacggtaaacttaaggttgtttccacagcaaaccaagataacccaatttctgatggccaaattccagtgcttggtctt

SEQ ID NO: 19 - DNA, 438 bp, Enterococcus sulfureus
caaatcgatgtggaaacaatgcatttacatcacgataaacatcacaatacttatgtgacgaacttaaatgcagcggttgaaaaatatcctgaattagcagaaaaatcagtggaagacttaatcgcagatatggatgcaatcccaagtgatattcaaacagcagtacgtaataatggtggtggccatgccaatcatagtttcttctgggaaatcttgacaccaaatgctactgaagaaccagtaggcgaattaaaaacagcgatcgaagatacatttggatctttagatgcattaaaagaagaatttaaaaaagcagcaactggccgttttggttcaggttgggcttggttagtagtaaaagacggtaaattagccgtaacgtctacagcaaaccaagattcaccattaatagaaggccaaactcctgttttaggttta

SEQ ID NO: 20 - DNA, 438 bp, Lactococcus garvieae
ttctttgatgaagaaacaatgcacttgcaccatgacaaacatcaccaaacatacgtaaataatcttaatgcagcgattgaaaaacacccagaattctttgataaaactgttgaagaattagtggcttatttggaccgtttgccagaagacattcgtgttgcagtacgtaacaacggtggaggacacttgaaccacacaatgttctgggaatggctcgctccaaatgcaggtggtgcaccaacaggtgatatcgctgcagcaatcgatgaagcttttggttcatttgacgacttcaaagctgaatttaaagcagctgctacaggacgtttcggttcaggttgggcttggttagttcttgattacggtaaacttaaagttgtttccacagcaaaccaagataacccaatttctgatggccaaattccagtgcttggtctt

SEQ ID NO: 21 - DNA, 438 bp, Enterococcus faecalis
tacattgacgtggaaacaatgcacttacaccatgataaacaccacaacacttatgtgactaacttaaacgcagcgattgaaaaacatccagaattaggcgaaaaatctgtagaagacctaatttcagatatgaatgctattcctgaagatatccgcacagccgttcgtaacaatggtggcgggcacgcaaaccatacattcttctgggaaattatggcaccaaatgctggtggacaaccaactggcgctattaaagaagcaatcgatgaaacatttggcagctttgatgaaatgaaagctgctttcaaaacagctgcaactggccgctttggttcaggttgggcttggttagttgtgaataacggtaaattagaaatcacttctacaccaaaccaagattcaccattaatggatggccaaacacctgttttaggtctt

SEQ ID NO: 22 - DNA, 438 bp, Enterococcus faecalis
tacattgacgtggaaacaatgcacttacaccatgataaacaccacaacacttatgtgactaacttaaacgcagcgattgaaaaacatccagaattaggcgaaaaatctgtagaagacctaatttcagatatgaatgctattcctgaagatatccgtacagccgttcgtaacaatggtggcggtcacgcaaaccatacattcttctgggaaattatggcaccaaatgctggtggacaaccaactggcgctattaaagaagcaatcgatgaaacatttggtagctttgatgaaatgaaagctgctttcaaaacagctgcaactggccgctttggttcaggttgggcttggttagttgtgaataacggtaaattagaaatcacctcaacaccaaaccaagattcaccattaatggatggccaaacacctgttttaggtctt

SEQ ID NO: 23 - DNA, 438 bp, Enterococcus faecalis
tacattgacgtggaaacaatgcacttacaccatgataaacaccacaacacttatgtgactaacttaaacgcagcgattgaaaaacatccagaattaggcgaaaaatctgtagaagacctaatttcagatatgaatgctattcctgaagatatccgcacagccgttcgtaacaatggtggcgggcacgcaaaccatacattcttctgggaaattatggcaccaaatgctggcggacaaccaactggcgctattaaagaagcaatcgatgaaacatttggcagctttgatgaaatgaaagctgctttcaaaacagctgcaactggccgctttggttcaggttgggcttggttagttgtgaataacggtaaattagaaatcacttctacaccaaaccaagattcaccattaatggatggccaaacacctgttttaggtctt

SEQ ID NO: 24 - DNA, 438 bp, Enterococcus durans
tatatcgatgaagaaacgatgcacttgcatcatgacaaacaccataatacttatgttacaaatttaaacgcagctattgaaaagtatccagaattaggcgaaaaatcagtggaagaattgctttctgatatggacgcgattcctactgatattaagacagcggtacaaaacaatggtggtggacatgcaaaccattcatttttctggaaaatcatggcacctaatgcaggtggcgaaccaacaggggaaatcaaagaagcgattgatgaagcttttggtgatttcgcaacatttaaagaagagttcaagaaagcggctgccggacgctttggatcaggttgggcatggttagtattggaagatggtaaattggcaatcacttctacagcaaaccaagattctccattgatgacaggccaaacacctatcttaggatta

SEQ ID NO: 25 - DNA, 438 bp, Enterococcus durans
tatatcgatgaagaaacgatgcacttgcatcatgacaaacaccataatacttatgttacaaatttaaacgcagctattgaaaagtatccagaattaggcgaaaaatcagtggaagaattgctttctgatatggacgcgattcccactgatattaagacagcggtacaaaacaatggtggtggacatgcaaaccattcatttttctggaaaatcatggcacctaatgcaggtggcgaaccaacaggcgaaatcaaagaagcgattgatgaagcttttggtgatttcgcaacatttaaagaagagttcaagaaagcggctgccggacgctttggatcaggttgggcatggttagtattggaagatggtaaattggcaatcacttctacagcaaaccaagattctccattgatgacaggccaaacacctatcttaggatta

SEQ ID NO: 26 - DNA, 438 bp, Enterococcus durans
tatatcgatgaagaaacgatgcacttgcatcatgacaaacaccataatacttatgttacaaatttaaacgcagctattgaaaagtatccaaaattaggcgaaaaatcagtggaagaattgctttctgatatggacgcgattcctactgatattaagacagcggtacaaaacaatggtggtggacatgcaaaccattcatttttctggaaaatcatggcacctaatgcaggtggcgaaccaacaggcgaaatcaaagaagcgattgatgaagcttttggtgatttcgcaacatttaaagaagagttcaagaaagcggctgccggacgctttggatcaggttgggcatggttagtattggaagatggtaaatcggcaatcacttctacagcaaaccaagattctccattgatgacaggccaaacacctatcttaggatta

SEQ ID NO: 27 - DNA, 438 bp, Enterococcus hirae
tatatcgatgaagaaacgatgcacttgcatcatgacaaacaccataatacttatgtaacaaatttaaatgcagcgattgaaaaacatccagaactaggtgaaaaaacaatcgaagaactactttctgatatggatgctgtccctacagatatcaagactgctgtacgtaataatggtggcggacatgcaaaccattctttcttctggaaaatcatggcaccaaatgctggtggcgaaccaactggtgcaattaaagaagcgattgatgaagcctttggtgattttgcaacatttaaggaagaatttaaaaaagctgcagctggccgttttggttcaggttgggcttggttagtgatggaaaatggtaaattagcgatcacttcaacagccaaccaagattcaccattaatggaaggcaaaacacctattttaggttta

SEQ ID NO: 28 - DNA, 438 bp, Enterococcus hirae
tatatcgatgaagaaacgatgcacttgcatcatgacaaacaccataatacttatgtaacaaatttaaatgcagcgattgaaaaacatccagaactaggtgaaaaaacaatcgaagaactactttctgatatggatgctgtccctacagatatcaagactgctgtacgtaataatggtggcggacatgcaaaccattctttcttctggaaaatcatggcaccaaatgctggtggcgaaccaactggtgcaattaaagaagcgattgatgaagcctttggtgattttgcaacatttaaggaagaatttaaaaaagctgcggctggccgttttggttcaggttgggcttggttagtgatggaaaatggtaaattagcgatcacttcaacagccaaccaagattcaccattaatggaaggcaaaacacctattttaggttta

SEQ ID NO: 29 - DNA, 438 bp, Enterococcus casseliflavus
tatattgatgaagaaacgatgcatttgcatcatgataaacaccacaacacttatgtaacaaacttaaatgcagcgattgaaaaacatcctgaattaggtgaaaaaacagttgaagaattattagcagacttttcttctgtacctgaagatattcaaacagcggttcgcaacaatggcggcggccatgctaaccacacattcttctgggaaatcttaggcccaaatgctggtggcgaacctactggggcaatcaaagaggcaattgaagaaacattcggcagctttgaagactttaaagaagaatttaaaactgctgcaactggacgttttggttcaggttgggcatggttagtcgttaaagacggtaaactagcaatcacttcaaccgcgaatcaagattcaccattgatggatggtcaaacacctgtattaggttta

SEQ ID NO: 30 - DNA, 438 bp, Enterococcus faecium
tatattgacgaagaaacgatgcatctgcatcatgataagcatcacaatacttatgtgacgaatttaaatgcagcaattgagaaatacccagaattaggcgaaaaaacaatagaagaattattatctgatatggacgctattccaacagatatcaagacagctgtacgtaacaatggtggcggacatgctaaccattcatttttctgggaaatcatggcaccaaatgctggtggcgaacctacaggagaaataaaagaagcgattaaagaagcttttggtgatttttcttcttttaaagaagaattcaaaaaagcagccgctggacgatttggttctggatgggcttggcttgtaatggaaaatggaaaattagctattacctctactgcaaatcaagattctccattgatggaaggaaagacaccaattctaggtttg

SEQ ID NO: 31 - DNA, 438 bp, Enterococcus faecium; identical to SEQ ID NO: 8

SEQ ID NO: 32 - DNA, 438 bp, Enterococcus faecium
tatattgacgaagaaacgatgcatctgcatcatgataagcatcacaatacttatgtgacgaatttaaatgcagcaattgagaaatacccagaattaggcgaaaaaacaatagaagaattattatctgatatggacgctattccaacagatatcaagacagctgtacgtaacaatggtggcggacatgctaaccattcatttttctgggaaatcatggcaccaaatgctggtggcgaacctacaggagaaataaaagaagcgattaatgaagcttttggtgatttttcttcttttaaagaagaattcaaaaaagcagccgctggacgatttggttctggatgggcttggcttgtaatggaaaatggaaaattagctattacctctactgcaaatcaagattctccattgatggaaggaaagacaccaattctaggtttg

SEQ ID NO: 33 - DNA, 438 bp, Enterococcus faecium; identical to SEQ ID NO: 32

SEQ ID NO: 34 - DNA, 438 bp, Enterococcus faecium
tatattgacgaagaaacgatgcatctgcatcatgataagcatcacaatacttatgtgacgaatttaaattcagcaattgagaaatacccagaattaggcgaaaaaacaatagaagaattattatctgatatggacgctattccaacagatatcaagacagctgtacgtaacaatggtggcggacatgctaaccattcatttttctgggaaatcatggcaccaaatgctggtggcgaacctacaggagaaataaaagaagcgattaatgaagctttttgtgatttttcttcttttaaagaagaattcaaaaaagcagccgctggacgatttggttctggatgggcttggcttgtaatggaaaatggaaaattagctattacctctactgcaaatcaagattctccattgatggaaggaaagacaccaattctaggtttg

SEQ ID NO: 35 - DNA, 438 bp, Enterococcus gallinarum
tgcattgatgaagaaacgatgcatttgcatcatgacaagcatcacaatacttacgtcacaaatttgaatgcagcaattgaaaaacatcctgaattaggtgaaaaatcagttgaagaattacttgctgattttgattcggtgcctgaagacatcaaaacagctgtccgtaataacggtggtggtcatgcaaatcacagctttttctgggaaatcttggcaccaaatgctggtggtgaaccaacaggagccatcaaagaagccatcgaagaaacatttggcagctttgctgatttcaaagaagaattcaaaacagcagcaactggccgctttggttctggctgggcttggttagtcatcaaagatggtaaattagcgatcacttcaactgcgaaccaagattcaccattaatggacggtcaaacgccagttttaggctta

SEQ ID NO: 36 - DNA, 438 bp, Enterococcus avium; identical to SEQ ID NO: 1

SEQ ID NO: 37 - DNA, 435 bp, Streptococcus difficilis
catattgatgctgagacaatgacactacatcatgataagcaccatgcaacttatgttgctaatgcaaatgctgctcttgagaaacatcctgaaattggagaagacttagaggcgctcttagctgatgtttctcaaattccagaagatattcgtcaggcagtcatcaataacggtggtggacatcttaaccacgctcttttctgggaattgatgtcaccagaagaaactcaaatttcaaaagagttatctgaagacattgatgcaacttttggttcatttgaagactttaaagctgctttcacagcagcagcaacaggacgttttggttcaggttgggcttggcttgttgttaatgctgaaggcaaacttgaagtgctttcaactgccaatcaagatactccaattatggaaggtaagaaaccaattttagggctt

SEQ ID NO: 38 - DNA, 435 bp, Streptococcus ferus
caaattgatgcggagacaatgactctccaccatgataaacaccatgcaacttatgtggctaacgcaaatgcagcccttgaaaaacacccagaaatcggtgacgatttagaaaaattgttggccgatgttgagtctattccagaagatatccgccaggctttgattaataatggcggaggccatctgaatcatgcgcttttctgggagttgctgtcaccagaaaaaacaaccatttcagctgaactgaaggctgatattgaagctagttttggttcttttgaccagtttaaagaggcctttacaacggctgctacaacacgctttggttcaggctgggcttggctcgttgtcaatcaagaaggacagttagaggtggtttcaacagctaatcaagacacaccaatttcacaaggtttgaaacccatcttggttcta

SEQ ID NO: 39 - DNA, 435 bp, Streptococcus gallolyticus
tatattgatacagaaacaatgacaattcaccatgataaacatcacgctacttatgtggcaaatgtaaatgcagcgcttgaaaaacatccagaaattggagaggatttggaagctttgttggcagatgttgacagtattccagcagatatccgtcaagcggtgattaataacggtggtgggcatttgaatcacgcccttttctgggaattgttatcgcctgaaaaacaagaaccaacagcgcaagtgttggctgcgattgaggaagattttggctcatttgacgaattcaaagctgctttcacgcaagctgcgacaactcgctttgggtcagggtgggcttggcttgtggtgaatgaaaatggcaaacttgaagtgctctcaacagctaatcaagacacaccaatttcacaaggaaaagcaccaattttggcactt

SEQ ID NO: 40 - DNA, 435 bp, Streptococcus hyointestinalis
tatatcgatgctgagacaatgactctccaccatgacaaacaccatgcgacttatgtggcaaatgtcaatgcggcccttgaaaaacacactgaaatcggtgaagacttggtggcacttttgtctgacgtggaaaaaatccctgctgacatccgtcaagccgttatcaacaacggcggaggacatctcaaccacgctcttttctgggaattgatgacaccagaaaagacagaggtttcagcagaattgttagcagatattgaagctacttttggctcatttgacgctttcaaagacgctttctcagcagcagctgcgactcgctttggctcaggttgggcttggcttgtcgtgaatgctgaaggaaaactcgaaattctctcaacagctaaccaagacaaccctatcatggatggcaaacaacctatccttggacta

SEQ ID NO: 41 - DNA, 435 bp, Streptococcus hyovaginalis
caaattgatgcagaaacaatgacccttcatcacgacaagcaccacgctacttatgtagcaaatgctaatgccgctttggaaaaacaccccgaacttggagatgacgttgcagcactcttatcggatgttgacagcattccagaagatattcgccaagccctcatcaataatggcggtggtcaccttaaccacgcattgttctgggaacttctttcaccagaaaagacagaaatcacagaagatgtcaaggctgctattgatgacgcttttggttcatttgacgccttcaaagaggcctttacggcggcagcaacaacacgttttggttcaggttgggcatggttagttgttaatgcagaaggaaaacttgaggtgacatcaactccaaaccaagatactccacttatggatggtaacacgccaatccttggttta

SEQ ID NO: 42 - DNA, 435 bp, Streptococcus infantarius
tacatcgatgcagaaacaatgacattgcatcatgacaaacatcacgctacttacgtagcaaatgcaaatgctgctcttgaaaaacaccctgaacttggagatgatttagaagttatcttggcagagcttgacaagattccagcagatattcgtcaagcggtgattaacaacggtggtggtgctcttaaccactcacttttctgggaattgctatctcctgaaaaacaagaaccaacagcagatgtacttgcggcaattgaagaagcatttggctcatttgaagatttcaaaacagctttcacgcaagcagcgacaactcgctttggttcaggttgggcttggcttgtcgttaacaaagatggcaaacttgaagtaacctcaactgctaaccaagatactccactttcagaaggtaagaaaccaattcttgctctt

SEQ ID NO: 43 - DNA, 435 bp, Streptococcus macacae
tattttgataaagaaacaatgacgcttcaccatgataaacatcatgccacttatgttgctaatgctaatgctgcattggaaaaacacccagaaataggtgaagatttagaaggcttactggcagatgttgagaagattcctgaggatattcgtcaggctttgattaataatggcggcggtcatcttaaccactctcttttttgggaattgctttccccagaaaaaacagaaatcactgaagaagtggctgcagctattaatgattcttttggctcttttgacgcttttaaagaagcatttacaactgctgcgacgactcgctttggttctggctgggcttggctggttgtcaaccgccaagggaagcttgaagtgatttcaacggctaatcaagatacgccaatttcacaagggctaaagccaatcctagcgctt

SEQ ID NO: 44 - DNA, 435 bp, Streptococcus macedonicus
tatattgatgcagaaacaatgacaattcatcatgataaacatcacgctacttatgtggcaaatgtaaatgcagcgcttgaaaaacatccagaaattggagaggatttggaaactttgttggcagatgttgacagtattccagcagatatccgtcaagcggtgattaataacggtggtgggcatttgaatcacgcccttttctgggaattgttatcgcctgaaaaacaagaaccaacagcgcaagtgctggctgcgattgaggaagcttttggctcatttgacgaattcaaagctactttcacgcaagctgcgacaactcgttttgggtcaggttgggcttggcttgtggtgaatgaaaatggcaaacttgaagtgctctcaacagctaatcaagatacaccaatttcacaaggaaaagcaccaattttggcactt

SEQ ID NO: 45 - DNA, 435 bp, Streptococcus parauberis
caatttgaccaagaaacaatgactctccatcatgataaacaccatgcaacttatgttgcaaatgccaatgctgctttagaaaaacacccagaaattggtgaagatctagaaactcttctagcagacgtggaatctattccttcagatattcgtcaagccctaattaataatggtggtggacatttgaatcacgcactattttgggaattattatctcctgagaatactgaaatttcttcagaagttgcatctgcaattgatgaagcatttggttcatttgatgcctttaaagaacaattcacagctgcagcaacaggacgttttggttctggatgggcatggctagttgtaaataaagaaggtaaacttgaaattatgtcaactgctaatcaagatacaccaatttcatcaggattaaaaccaattttaggattg

SEQ ID NO: 46 - DNA, 435 bp, Streptococcus phocae
tattttgatatggagacaatgactctgcatcatgacaagcaccatgcaacatatgttgcaaacactaatgctgctttggaaaaacaccctgaaatcggtgaggaccttgaagcattgttagcagatgttgatgcgataccagcagatattcgtcaagctgtgataaataacggtggtgggcatttgaatcatagcttgttctgggaattactgtctccagaaaagcaagaggttactgctgacgttgccgcagccattgacgaagcatttggttcgtttgatgcttttaaagaacaattcactgcagcagcaacaggtcgctttggatcaggttgggcatggttagttgtcaataaagaaggcaagcttgaaatcacgtcaactgctaaccaagacacaccaatctcagatggtaaaaagcctattttaacgctt

SEQ ID NO: 47 - DNA, 435 bp, Streptococcus ratti
tatattgatgcagaaacaatgacccttcatcatgataaacaccatgctacctatgtggcaaatgctaatgcagctctcgaaaaacatccagaaattggtgaaaatttagaagttctcttggctgatgttcagcaaattccggaagatatccgtcaggctcttgttaacaacggcggcggtcaccttaaccacgcacttttctgggaacttctgtcaccagaaaaaacagagattactaaagaagtggctgcagcaattgacgaagcttttggctcatttgaggcttttaagacagctttcactcaggcagcagcaacacgctttggttcaggctgggcttggctagttgtcaacgcagaaggtaagcttgaagtaatgtcaacagccaaccaagatacaccgatttcgcaaggtttaaaaccaatcttggccctt

SEQ ID NO: 48 - DNA, 435 bp, Streptococcus thoraltensis
cattttgatgcggagacaatgactcttcaccatgataaacaccatgcgacatacgtgaacaacgcaaatgctgctttggaaaaacaccctgaaatcggtgaagaccttgaagctcttttgtcagatgtcaacagcattcctgaagacattcgtcaagcgcttatcaacaatggcggtggacatcttaaccatgcccttttctgggaacttctttcaccagaaaaaacagaaattacagaagatgtgaaagcagccattgatgaagctttcggttcatttgaagccttccaagaaaaattcactacagcagctacaacacgctttggttcaggttgggcttggttagttgttaacgctgaaggtaaactcgaggtcacatcaacaccaaaccaagacactccacttatggaaggtaaaaaaccaatccttggactt

SEQ ID NO: 49 - DNA, 435 bp, Streptococcus uberis
caaattgataaagaaacaatgactcttcatcatgacaaacatcatgcgacatatgttgctaatgccaatgctgcgcttgaaaaacatccagaaattggtgaagatttggtggcgttattatctgatgtgtcatcaattccagaagatattcgtcaagctcttatcaataatggaggcggacatcttaaccatgcacttttttgggaacttctttcacctgagaaaacagaaatcacttcggaagtagcttctgctattgatgaagcatttggttcttttgatgcatttaaagaaaaatttacagcagcagcaacgggacgttttggatctggttgggcttggttagttgtcaataaagaaggagaacttgaagtaacttcaactgcaaaccaagatacaccaatttctgaaggtaaacagcctattttgggtctt

SEQ ID NO: 50 - DNA, 435 bp, Streptococcus waius; identical to SEQ ID NO: 44

SEQ ID NO: 51 - DNA, 438 bp, Abiotrophia adiacens
cattttgatgcacgtacaatggaaatccaccatgacaaacatcacaatgcatatgttacaaatttaaacgcagcggtagaaaaacaccctgaattattcgaaaaaacagttgaagaattagttagcgatttaaacgctgttccagaagatatccgtgtagctgttcgcaacaatggtggtgggcatgcaaaccatagcttattctggactcaattatctcttgatggtgcaaaagctccagaaggtgctttattagcagctatcaacgaagcattcggaagcttcgacgaattcaaagcagcattcgcacaagcagcagcaactcgttttgggtctggttgggcttggttagttctttctaacggaaaattagaagtcgtttctactccaaaccaagataaccctctatcagaaggcaaaactccattattaggatta

SEQ ID NO: 52 - DNA, 441 bp, Abiotrophia defectivus
gcttttgacgcgcgcaccatggaaattcaccacaccaagcaccaccaaacccacgttaacaacttgaatgccgccttagaaggtcacgcagacttggcagctaagtctatcgaagacttagtcgctaaccttaaggatttacctgaaagcattcaaacagctgtccgtaacaatggtgggggtcacttcaaccatagcttcttctgggaaagcctacaagcgccaagtgcagaagcagctattcctgctggcctcaagtctcgcttagaagcagactttggttctgttgaagccttcaaagaagcttttgctaaggcagctgcgactcgctttggttctggttgggcttggctcgtagaccgtgacggtcacttagaagtcttatctactgctaaccaagacacacctttagaattagggcttaagccacttttaggttta

SEQ ID NO: 53 - DNA, 438 bp, Abiotrophia elegans
catgtggatgctttaacaatggaaatccatcatgacagacatcataacacttatgtaacaaacttaaacgcagcagtagaaaaacaccctgctttatttgaaaaaagtgtggaagaattagtagcagatttagcatctgtaccagaaggtattcgtggagctgttcgtaacaatggtggtggacatgcaaaccacagcttattctggacagtaatttcaccgaatggtggaggtcaacctactggcgaattagcagcagcaatcgatagcaaattcggtgggtttgatgcgtttaaacaagcattctctcaagcagcagcaactcgtttcggttctggttgggcttggttagttgtttcaaatggtgaattagaagtagtttctactccaaaccaagataacccattaacagatggtaaaactccaattttaggatta

SEQ ID NO: 54 - DNA, 429 bp, Staphylococcus arlettae
cacattgataaagaaacaatggaaattcatcatgacaagcaccacaacacatatgttacaaaattaaatgcagcagtagaaggtactgatttagaatctaaatcaattgaagaaatcgtcgctaacttagatagcgtacctgaagatattcaaacagctgtgcgtaacaatggtggaggacatatcaaccattcattgttctgggaattattaactcctaactctgaagaaaaaggtactgtagttgataaaattaaagaacaatggggttctttagatgcatttaaagaagaatttgcaaataaagctgcagcacgttttggttcaggttgggcatggttagtagtaaataacggtaacttagaaatcgttactacacctaaccaagacaacccattaactgaaggtaaaacacctattttaggttta

SEQ ID NO: 55 - DNA, 432 bp, Staphylococcus auricularis
tatattgataaagaaactatggaaatccaccatgacaaacaccacaacacatatgtaactaaattaaattcagcagttgaaggtacagatttagaaaataaatctatcgaagaaattgttgctaatttagatagcgtacctgaagatattcaaacagctgtacgaaataatggtggtggacacttaaatcactcattattctgggaattattaactcctaactctgaagaaaaaggtacagtcgtagataaaattaaagaacaatggggttctttagacgatttcaaaaaagaatttgctgacgctgcagcagctcgctttggttcagactggggttggctcgttgtaaatgctgaaggtaaattagaaatcactactacacctaaccaagataacccaattacagaaggtaaaacacctattttaggtatt

SEQ ID NO: 56 - DNA, 429 bp, Staphylococcus capitis subsp. captis
cacattgataaacaaactatggaaattcaccatgacaaacaccataacacatatgtaactaaattaaactcagcagttgaaggaacagatttagaagctaaatcaatcgaagaaattgttgctaatttagatagcgtaccttcagatattcaaactgcagtacgtaataatggtggcggtcacttaaaccactcattattctgggaattattatcaccaaattctgaagaaaaaggtgaagtagtagacaaaattaaagaacaatggggttctttagatgaattcaaaaaagaatttgcagataaagctgctgcacgctttggatctggttgggcatggttagtagtaaataacggtcaattagaaatcgttactactccaaaccaagataacccattaactgaaggtaaaactccaatcttaggttta

SEQ ID NO: 57 - DNA, 429 bp, Staphylococcus capitis subsp. ureolyticus
cacattgataaacaaactatggaaattcaccacgacaaacaccataacacatatgtaactaaattaaactcagcagttgaaggaacagatttagaagctaaatcaatcgaagaaattgttgctaatttagatagcgtaccttcagatattcaaactgcagtacgtaataatggtggcggtcacttaaaccactcattattctgggaattattatcaccaaattctgaagaaaaaggtgaagtagtagacaaaattaaagaacaatggggttctttagatgaattcaaaaaagaatttgcagataaagctgctgcacgctttggatctggttgggcatggttagtagtaaataacggtcaattagaaatcgttactactccaaaccaagataacccattaactgaaggtaaaactccaatcttaggttta

SEQ ID NO: 58 - DNA, 429 bp, Staphylococcus caprae
cacatcgataaacaaactatggagattcatcacgacaaacaccataacacatatgtaactaaattaaactcagcagttgaaggaacagatttagaagctaaatcaatcgaagaaattgttgcaaatttagatagcgtaccttctgatattcaaacagcagtacgtaacaatggtggcggtcacttaaaccactcattattctgggaattattatcacctaattctgaagaaaaaggtgaagttgtagacaaaatcaaagaacaatggggctctttagatgaattcaaaaaagaattcgctgacaaagcagcagctcgtttcggttcaggttgggcttggttagtagtaaacaacggtcaattagaaatcgtaactacaccaaaccaagataacccattaactgaaggtaaaacaccaatcttaggttta

SEQ ID NO: 59 - DNA, 432 bp, Staphylococcus carnosus subsp. carnosus
tatatcgataaagaaacaatggaaatccatcatgacaaacatcataatacttatgtaacaaaattaaatgcagcaatcgaaggtactgatttagaaaataaatctatcgaagagatcgttgctaatttagacagcgtaccatctgacatccaaactgcagttcgtaataacggtggtggacatttaaaccattcattattctggcaacttctaacacctaattctgaagaaaaaggtacagtaattgataaaatcaaagaagaatggggttctttagacaaatttaaagatgaatttgctaaaaaagctgctggacaatttggttcaggttgggcatggctagttgtagataaagacggtaaactagaaatcgtttctactcctaaccaagacaatccaatcacagaaggcaaaactcctattttaggactt

SEQ ID NO: 60 - DNA, 432 bp, Staphylococcus carnosus subsp. utilis
tatatcgataaagaaacaatggaaatccatcatgacaaacatcataacacttatgtaataaaattaaatgcagcaatcgaaggtactgatttagaaaataaatctatcgaagagatcgttgctaatttagacagcgtaccatctgacatccaaactgcagttcgtaataacggtggtggacatttaaaccattcattattctggcaacttctaacacctaattctgaagaaaaaggtacagtaattgataaaatcaaagaagaatggggttctttagacaaatttaaagatgaatttgctaaaaaagctgctggacaatttggttcaggttgggcatggctagttgtagataaagacggtaaactagaaatcgtttctactcctaaccaagacaatccaatcacagaaggcaaaactcctattttaggactt

SEQ ID NO: 61 - DNA, 429 bp, Staphylococcus chromogenes
catattgataaagaaacgatggaaatccatcatagtaaacaccataacacatacgtgactaagttaaacgatgcagttaaaggtactgatttagagaacaaatcaatcgaagaaattattgctaacttaaatagcgtaccagaagataaacaaactcctgtacgtaataatggtggcggtcacttaaaccactctttattctggcaattactttcaccacaatcagaagaaaaaggtgaagtcgtagataaaattaaagagcaatggggctctttagatgatttcaaaaaagaatttgcagacaaagcagcagctcgttttggttctggttgggcatggctcgttgtaaataatggtcaattagaaatcgttactacaccaaaccaagacaacccaatttctgaaggtaaaactcctatcttaggatta

SEQ ID NO: 62 - DNA, 429 bp, Staphylococcus cohnii subsp. cohnii
catattgatcaacaaacaatggaaattcatcacgacaaacatcataacacttatgttactaaattaaatgcagcaattgaaggtactgatttagagtctaaatcaattgaagaaattattgcaaatttagacagtgtaccagaagatattcaaacagctgttagaaataatggcggtggacacttaaaccactcattattctgggaattattaactccaaactctgaagaaaaaggaactgtagttgataaaattaaagaacaatggggttctttagatgcatttaaagaagaatttgcagataaagctgcagctcgttttggttcaggatgggcttggctagttgttaataatggtaatttagaaattgttacaactccaaaccaagataacccacttacagaaggtaaaacaccaatcctaggctta

SEQ ID NO: 63 - DNA, 429 bp, Staphylococcus cohnii subsp. urealyticum
catattgatcaacaaacaatggaaatccaccatgacaaacatcataacacttatgttactaaattaaatgcagcaattgaaggtactgatttagaatctaaatcaattgaagaaattgtagcaaatttagacagtgtaccagaaaatattcaaacagctgttagaaataatggtggtggacacttaaaccattcattattctgggaattattaactccaaactctgaagaaaaaggaactgtagttgataaaattaaggaacaatggggttctttagatgcatttaaagaagaatttgcagataaagctgcagctcgttttggttcaggttgggcttggctagttgttaataatggcaatttagaaattgttacaactccaaaccaagataacccattaactgaaggtaaaacacctatcttaggctta

SEQ ID NO: 64 - DNA, 432 bp, Staphylococcus condimenti
tatatcgataaagaaacaatggaaatccatcatgacaaacatcacaacacttatgtaacaaaattaaatgcagcaatcgaaggtactgatttagaaaataaatctatcgaagaaatcgttgcaaatttagacagcgtaccatctgacatccaaactgcagttcgtaataatggtggtggacatctaaaccattcattattctggcaacttctaacacctaattctgaagaaaaaggtacagtaattgataaaatcaaagaagaatggggttctttagacaaattcaaagatgaatttgctaaaaaagctgctggacaatttggttcaggttgggcttggctagttgtagataaaaacggtaacttagaaatcgtttctactccaaaccaagacaacccaattacagaaggcaaaactcctattttaggactt

SEQ ID NO: 65 - DNA, 429 bp, Staphylococcus delphini
cacattgataaagaaactatggaaatccatcacagcaagcatcataacacttatgtaacaaaattaaatgctgctgttgaaggtactgaatttgaaaataaatcattagaagatttaattgcaaacttagacagcgtaccagaaaacttacgtacagcagttcgtaataatggtggcggtcacttaaatcactctattttctggcaaatcttaacacctaactcagaagaaaaaggtgaagttgtcgataaaattaaagaacaatggggttctttagatgaattcaaaaacgaatttgcagacaaagcagctggccgtttcggttcaggttgggcttggcttgttgttaacaacggtaaattagaaatcgttacaactgcaaaccaagatagtccattaactgatggtttaacaccaattttagcgtta

SEQ ID NO: 66 - DNA, 429 bp, Staphylococcus epidermidis
cacatcgacaaacaaactatggaaattcatcatgacaaacatcataacacatatgttacaaaattaaattcagcagttgaagggacagatttagaagctaaatcaatcgaagaaattgttgctaatttagatagtgtgccatctaatattcaaacagctgttcgtaataatggcggtggtcaccttaaccattcattgttctgggaactattatcaccaaattctgaagaaaaaggtgaagtagtagataaaattaaagaacaatggggttctttagatgaatttaaaaaagaatttgcagataaagctgcagcacgctttggttcaggatgggcttggttagttgtaaacaatggacaattagaaattgttacaacaccaaatcaagataatccaattactgaaggaaaaacaccaattttaggttta

SEQ ID NO: 67 - DNA, 429 bp, Staphylococcus equorum
cacattgatcaacaaacaatggagattcaccatgacaaacaccataacacttatgtaactaaattaaacgcagcagttgaaggaactgatttagaatctaaatcaatcgaagaaattgttgcaaacttagacagtgtaccagaaaacattcaaacagctgttcgcaataatggtggaggacacttaaaccattcattattctgggaattattaactccaaactctgaagaaaaaggtactgttgttgataaaattaaagaacaatggggttctttagatgcattcaaagaagagtttgctaaccaagctgcagcacgtttcggttcaggttgggcatggctagttgtaaacgatggtaaattggaaatcgttactacacctaatcaagataacccattaactgaaggtaaaacacctatcctaggctta

SEQ ID NO: 68 - DNA, 429 bp, Staphylococcus felis
cacatcgataaagaaactatggaaattcaccatagcaaacaccataacacatatgttacaaaattaaacgctacagtagaaggttcagatttagaaaataaatctcttgaagatcttattgccaatgtagatagtcttccagaagacaagaaaacagctgtacgtaataatggtggcggtcatcttaaccactcattcttctgggcacttttaacacctaattctgaagaaaaaggtgaagtagttgataaaatcaatgaaaaatggggctcattagacgcattcaaaaaagaatttggcgatgcggctgctggtcgatttggttcaggctgggcatggttagttgtgaacaatggtgaattagaaattgtttcaacacctaaccaagacaatccattgtctgaaggtaaaacgccaattttagctctt

SEQ ID NO: 69 - DNA, 429 bp, Staphylococcus gallinarum
aatattgacaaagaaactatggaaatccaccatggtaaacaccacaacacttatgtaactaaattaaatgctgcagttgaaggtactgatttagaatctaaatcaatcgaagaaattgttgcaaacttagacagtgtaccagaaaatattcaaacagctgttagaaataatggtggtggacacttaaaccactcattattctgggaattattaactcctaactctgaagaaaaaggtactgtagttgataaaattaaagaacaatggggttctttagatgcatttaaagaagaatttgcagataaagctgcagcacgctttggttcaggttgggcatggctagttgtaaataacggtaacttagaaatcgttactacacctaaccaagacaaccctattactgaaggtaaaacacctatcttaggttta

SEQ ID NO: 70 - DNA, 429 bp, Staphylococcus haemolyticus
cacattgacaaacaaactatggaaatccatcatgacaaacaccacaacacgtatgttaccaaattaaattctgcagttgagggaacagatcttgaatctaaatcaattgaagaaattgttgctaatttagatagtgtacctgaagatattcaaacagctgttcgtaataatggtggcggacacttaaatcactcattattctgggaattattaactcctaattctgaagaaaaaggtactgttgttgataaaatcaaagaacaatggggctctttagatgaattcaaaaaagaattcgctgacaaagcagcagctcgtttcggttcaggttgggcatggttagtagttaacaatggtcagttagaaattgttactacacctaaccaagataacccattaacggaaggtaaaacacctatcttaggttta

SEQ ID NO: 71 - DNA, 429 bp, Staphylococcus hominis subsp. hominus
catatcgacaaagaaacaatggaaattcatcatgacaaacatcataacacttatgttacaaaattaaactctgcagttgaaggtactgatttagaatctaaatcaattgaagaaattgttgcaaatttagatagtgtatctgaaaatattcaaacagcagtacgtaataatggtggaggtcatttaaatcactcattattctgggaattattaactcctaattctgaagaaaaaggtactgtagttgataaaattaaagaacaatggggttctttagatgagtttaaaaaagaattcgctgataaagctgcagcacgttttggttcaggttgggcttggttagtagtaaataatggaaaattagaaattgttactactccaaatcaagataaccctattactgaaggaaaaactccaattttaggctta

SEQ ID NO: 72 - DNA, 429 bp, Staphylococcus hominis subsp. novobiosepticus
catatcgacaaagaaacaatggaaattcatcatgacaaacatcataacacttatgttacaaaattaaattctgcagttgaaggtactgatttagaatctaaatcaattgaagaaattgttgcaaatttagatagtgtacctgaaaatattcaaacagcagtacgtaataatggtggaggtcatttaaatcactcattattctgggaattattaactcctaattctgaagaaaaaggtactgtaattgataaaattaaagaacaatggggttctttagatgagtttaaaaaagaattcgctgataaagctgcagcacgttttggttcaggttgggcttggttagtagtaaataatggaaaattagaaattgttactactccaaatcaagataaccctattactgaaggaaaaactccaattttaggctta

SEQ ID NO: 73 - DNA, 429 bp, Staphylococcus hyicus
catattgacaaagaaactatggaaatccaccatagcaaacatcataacacttatgtaacaaaattaaacgacgctgtaaaaggtacagagttagaagataaatctattgaagagcttatcgcgaatgttgaccaattacctgaggataaaaagactgcggttcgtaacaatggtggcggtcactttaaccattctttattctggcaatttttatccccagaatctgaagaaaaaggtgaagttgttgacaaaattaaagaacaatggggttctttagacgcatttaaaaaagaattctcagataaagcagcagcacgatttggatctggctgggcttggcttgtagtaaataatggtcaattagaaattgttacaacagcaaaccaagatagcccattatcagaaggtaagacaccaatactcgctcta

SEQ ID NO: 74 - DNA, 429 bp, Staphylococcus intermedius
cacattgataaagaaactatggaaatccatcacagtaagcatcataacacttacgtaacgaaattaaatgctgctgttgaaggtactgaatttgaaaataaatcattagaagatttaattgcaaacttaaatagtgtacctgaaaacattcgtacagcggtacgtaataatggtggcggtcacttaaatcactctattttctggcaacttttaacacctaactcagaagaaaaaggtgaagttgtagataaaatcaaagaacaatggggttctttagatgaatttaaaaacgaatttgcggataaagcagcagcacgtttcggttcaggttgggcttggcttgttgtcaataacggcaaattagaaatcgttacaacagcaaaccaagacagtccattaactgacggattatcaccaatcttagcatta

SEQ ID NO: 75 - DNA, 429 bp, Staphylococcus kloosii
cacatcgataaagaaactatggaaattcaccacgataaacaccataacacttatgtaacaaaattaaacgcagcagttgaaggaactgaattagaatctaagtcaattgaagaaattattgcaaacttagacagtgttcctgaaaacattcaaacagctgttcgtaataatggtgggggacatattaaccattcattattctgggaattattaactcctaactctgaagaaaaaggtactgtagtagataaaattaaagaacaatggggttctttagatgcatttaaagaagaatttgctgataaagctgcaggccgtttcggttcaggttgggcatggttagtagtaaataacggtaacttagaaatcgttactacacctaaccaagacaatccattaactgaaggtaaaacacctatcttaggttta

SEQ ID NO: 76 - DNA, 432 bp, Staphylococcus lentus
cacatcgataaagagacaatggagattcatcatacgaaacaccataacacttatgtaacaaaactaaatgatgcagttaaaggtactgacttagaaagtaaatctattgaagatattattaaaaacttaaattctgtaccagatgatatccgtactgcagttcaaaacaatggtggcggacattacaatcactcattattctgggagatgttaactccaaatgcttctgaaccatcaggcgaagtagtagatgcaatcagttctactttcggttcattagacaaatttaaagaagagtttgcagcagcagcagctggacgcttcggttcaggttgggcatggttagttgtagataacggtgaattatcaatcgtttcaactccaaaccaagataacccattatctgaaggtaaaattcctgtattaggatta

SEQ ID NO: 77 - DNA, 429 bp, Staphylococcus lugdunensis
catattgataaagaaacaatggaaatccatcatgataaacatcataatacgtatgtgactaaattaaattctgcagttgaaggtacagacttagagtctaaatctattgaggaaattattgccaatttagatagcgttcctgaaaacattcaaacagctgtacgtaataatggtggtggacacttaaaccattcactattctgggaatttttaactcctaattctgaagaaaaaggtactgtagttgataaaattaaagaacaatggggttctttagatgaattcaagaaagaattcgctgacaaagctgcaggtcgttttggttcaggttgggcatggttagttgtaaataacggtaaattagaaattgttacaacgcctaaccaagacaacccattaactgaaggaaaaacacctatcttatgtata

SEQ ID NO: 78 - DNA, 429 bp, Staphylococcus lurae
catatcgataaagaaacaatggagctccatcacggtaaacatcataacacatacgttactaaattaaatgctgctgttgaaggcacagaattggaaaataaatcacttgaagatttaatcacacatttagatcgcgtacctgaaaatgtacgtactgctgtgcgtaacaatggtggcggtcatttaaatcactcatttttctggcaactgcttacaccaaactctgaagaaaaaggtgaagtagtggataaaattaaagaacaatggggatcattagacgcattcaaagaagaatttgcagataaagcagcgggtcgtttcggttctggttgggcttggcttgttttaaataatggaaaattagaaattattacaacacctaaccaagacagtccgttaactgaaggtttaacaccgcttttaacttta

SEQ ID NO: 79 - DNA, 429 bp, Staphylococcus muscae
catttcgacaaagaaacaatggagattcatcatacgaagcatcataacacttatgttacaaagttaaacggtgcagttgaaggaacagaatttgaaaacaaatcaattgaagatcttgttgcaaacttaaatgatgtacctgaagaaaaacgcacagctgtacgtaataatggtggcggccacttaaaccactcattattctggcagttattaacacctaattcagaagaaaaaggtacagtggttgaaaaaatcactgaaaaatggggtagcttagatagtttcaaacaagaatttgccgataaagcagcagctcgattcggttcaggttgggcatggttagttgtagacaatggcgagttagcgattgtgacaactccaaatcaagacaatccaatcacagatggaaaaactccactattaggtctt

SEQ ID NO: 80 - DNA, 429 bp, Staphylococcus pasteuri
cacatcgataaagaaactatggagattcaccatgataaacaccataacacttatgtaacaaaattaaacgctgcagttgaaggtactgatttagaagctaaatcaatcgaagaaattgtagctaatttagacagtgtaccttctgatatccaaactgctgttagaaataatggtggcggacacttaaaccactcattattctgggaattactaacacctaactcagaagaaaaaggtgaagtagtagataaaattaaagaacaatggggttctttagatgaattcaaaaaagaatttgctgacaaagcagcagctcgttttggttcaggttgggcttggttagtagtaaataacgggcaattagaaatcgttacaactccaaaccaagataatccattaactgaaggtaaaacacctatcttaggttta

SEQ ID NO: 81 - DNA, 432 bp, Staphylococcus piscifermentans
tatatcgacaaagaaacaatggaaatccatcatgacaaacaccataacacttatgtaacaaaattaaatgcagcaatcgaaggtactgatttagaaaataaatcaatcgaagaaatcgttgctaatttagacagcgtaccatcagacatccaaactgcagttcgtaataatggtgggggacacttaaaccactcattattctggcaacttcttacacctaattctgaagaaaaaggtactgtaattgataaaattaaagaagaatggggctctttagataaattcaaagatgaatttgctaaaaaagctgctggacaatttggttcaggttgggcatggctagttgtagataaaaacggtaaattagaaatcgtttctacaccaaaccaagacaaccctattacagaaggcaaaactcctattttaggctta

SEQ ID NO: 82 - DNA, 432 bp, Staphylococcus pulvereri
cacatcgataaagaaactatggagattcaccatacgaaacaccataacacttatgtaacaaaattaaatgacgcagttaaaggtacagatttagaaagtaaatctattgaagatatcattaaaaatttaaattctgttcctgaaaatattcgtactgcagttcaaaacaatggtggcggacattataatcactcattattctgggaactattaacaccaaatgcttctgaaccttcaggagaagttgtggatgcaattagttctacattcggttcattagacaaattcaaagaagaatttgcagctgcagcagctggccgtttcggttcaggttgggcatggttagttgtagataacggtgaattagcgatcgtttcaactccaaaccaaggtaaccctatttcagaaggtaaacttccagtattaggctta

SEQ ID NO: 83 - DNA, 429 bp, Staphylococcus saccharolyticus
cacatcgataaacaaactatggaattacatcatgacaaacatcacaacacatatgtaactaaattaaactcagcagttgaaggaacagatttagaagctaaatcaatcgaagaaattgttgctaatttagacaatgtcccatcaaatattcaaacagctgttcgtaacaatggcggtggacatttaaaccattcattattctgggaattattatcacctaactcagaagaaaaaggtgaagttgtagataaaattaaagaacaatggggttctttagatgaatttaaaaaagaatttgcggataaagctacagctcgttttggttcaggttgggcatggttagtagtagataatggccaattagagattgtaacaacacttaatcaagacaatccattaactgaagggaaaactccaattctagcttta

SEQ ID NO: 84 - DNA, 429 bp, Staphylococcus saprophyticus subsp. bovis
cacattgataaacaaacaatggaaattcaccatgacaaacaccataacacttatgtaactaaattaaatgcagcagtagaaggaactgatttagaatctaaatcaatcgaagaaattgttgcaaacttagacagtgttccagaaaatattcaaacagctgttcgaaataatggtggtggacacttaaaccactcactattctgggaattattaactccaaactcagaagaaaaaggtactgttgttgataaaattaaagaacaatggggctctttagatgcatttaaagaagaatttgctgacaaagcagcagctcgtttcggttcaggttgggcatggctagttgtgaataacggtaacttagaaatcgttacaacacctaaccaagataacccattaactgaaggtaaaacaccaatcttaggatta

SEQ ID NO: 85 - DNA, 429 bp, Staphylococcus saprophyticus subsp. saprophyticus; identical to SEQ ID NO: 84

SEQ ID NO: 86 - DNA, 429 bp, Staphylococcus saprophyticus subsp. coagulans
cacattgacaaagaaacaatggtgctacatcatgacaaacatcataatacgtatgtaacaaagttaaacgcagcagttgaaggtacagatttagaaaataaatctattgaagatttaattgctaatttagacagtgtgcctgaagataaacgtactgcagttcgtaataatggtggtggacacttaaaccactcatttttctggcaaattatttcacctaactcagaagaaaaaggtgaagttgtagataaaattaaagaacaatggggttctttagatgcattcaaaaaagaatttgctgataaagctgcaggtcaatttggttcaggttgggcatggttagtagtgaataatggtcaattagaaatcgtaacgactcctaaccaagatagtccacttactaatggccaaactccaattttaaactta

SEQ ID NO: 87 - DNA, 429 bp, Staphylococcus schleiferi subsp. schleiferi
cacattgataaagaaacaatggtgctacatcatgacaaacatcataatacgtatgtaacaaagttaaacgcagcagttgaaggtacagatttagaagataaatctattgaagatttaattgctaatttagatagtgtacctgaagataaacgtactgcagttcgtaataatggtggtggacacttaaaccactcatttttctggcaaattatttcacctaactcagaagaaaaaggtgaagttgtagataaaattaaagaacaatggggttctttagatgcattcaaaaaagaatttgctgaaaaagctgcaggtcaatttggttcaggttgggcatggttagtagtgaataatggtcaattagaaatcgtaacgactcctaaccaagatagtccacttactaatggccaaactccaattttaaactta

SEQ ID NO: 88 - DNA, 432 bp, Staphylococcus sciuri subsp. carnaticus
cacatcgataaagaaactatggagattcatcatacgaaacaccataacacttatgtaacaaaattaaatgatgcagtgaaaggtacagatttagaaagcaaatctattgaagatattgttaaaaacttaaactctgttcctgatgatatccgtactgcagttcaaaacaatggtggcggacattataatcattcattattctgggaactattaactccaaatgcttctgagccttcaggagaagttgtagatacaattagttctacatttggttcattagacaaattcaaagaagaatttgcagctgcagcagctggccgttttggttcaggatgggcatggttagttgtagataatggcgaattagcgattgtttcaactccaaaccaagataacccaatttcagaaggtaaacttccaattttaggttta

SEQ ID NO: 89 - DNA, 432 bp, Staphylococcus sciuri subsp. sciuri
cacatcgataaagaaactatggagattcatcatacgaaacaccataacacttatgtaacaaaattaaatgatgcagtgaaaggtacagatttagaaagcaaatctattgaagatattgttaaaaacttaaactctgttcctgatgatatccgtactgcagttcaaaacaatggtggcggacattataatcattcattattctgggaactattaactccaaacgcttctgagccttcaggagaagttgtagatgcaattagttctacatttggttcattagacaaattcaaagaagaatttgcagctgcagcagctggccgttttggttcaggatgggcatggttagttgtagataatggccaattagcgattgtttcaactccaaaccaagataacccaatttcagaaggtaaacttccaattttaggttta

SEQ ID NO: 90 - DNA, 432 bp, Staphylococcus simulans
cacatcgataaagaaacgatggaaattcaccatgacaaacaccataacacttatgttacaaaattaaacgcagcaatcgaaggaactgatttagaaaacaaatcaatcgaagaaattgttgctaacttagatagtgtaccatctgacatccaaactgcagtccgtaataatggtggtggacacttaaaccactcattattctggcaaatcctttcacctaactctgaagagaaaggtacagtagttgataaaattaaagaacaatggggttctttagacgaattcaaagacgaatttgctaaaaaagctgctggacaatttggttcaggttgggcttggctagtagtagataaagacggtaaattagaaatcgttactacagcaaaccaagacaacccaattactgaaggcaaaactcctatcctaggctta

SEQ ID NO: 91 - DNA, 432 bp, Staphylococcus vitulinus
cacatcgataaagaaactatggagattcaccatacgaaacaccataacacttatgtaacaaaattaaatgacgcagttaaaggtacagatttagaaagtaaatctattgaagatatcattaaaaatttaaattctgttcctgaaaatattcgtactgcagttcaaaacaatggtggcggacattataaccactcattattctgggaactattaacaccaaatgcttctgaaccttcaggagaagttgtggatgcaattagttctacattcggttcattagacaaattcaaagaagaatttgcagctgcagcagctggccgtttcggttcaggttgggcatggttagttgtagataacggtgaattagcgatcgtttcaactccaaaccaagataaccctatttcagaaggtaaacttccagtattaggctta

SEQ ID NO: 92 - DNA, 429 bp, Staphylococcus warneri
cacatcgataaagaaactatggagattcaccatgataaacaccacaacacttatgtaacaaaattaaatgcagctgtagaaggtactgacttagaagctaaatcaatcgaagaaattgtagctaacttagatagtgttccttctgatattcaaactgcagtaagaaataacggtggcggacacttaaaccactcattattctgggaattattaacacctaattcagaagaaaaaggtgaagtagtagataaaattaaagaacaatggggttctttagatgaattcaaaaaagaattcgctgacaaagctgcagctcgttttggttcaggttgggcttggttagttgttaataatggtcaattagaaatcgttacaactccaaaccaagataacccattaactgaaggtaaaacacctatcctaggctta

SEQ ID NO: 93 - DNA, 429 bp, Staphylococcus xylosus
cacattgatcaacaaacaatggaaattcaccatggcaaacaccataacacttatgtaactaaattaaacgcagcagtagaaggaactgatttagaatctaaatcaattgaagaaattgttgcaaatctagacagtgttccagaaaatattcaaacagctgttcgcaataatggcggtggacatttaaaccactcattattctgggaattattaactccaaactcagaagaaaaaggtactgttgttgataaaattaaagaacaatggggttctttagatgcatttaaagaagaatttgcagacaaagcagcagcacgtttcggttcaggttgggcctggttagtagttaataacggtaacttagaaatcgttactacacctaaccaagacaatccaattactgaaggtaaaacacctatcttaggatta

SEQ ID NO: 94 - DNA, 427 bp, Macrococcus caseolyticus
cacatcgacaaagaaacgatggagattcaccatacaaaacatcataacacttatgtaacgaaattaaatgatgcagtggctggtacggagtttgagaatgtatctatcgaagatctgatgaagagaattgatgaagttcctgcagacaagaaaactgctgtagttaataatggtggcggtcattataaccactcattgttctggacattgcttgctccaggtaaagaagcgaaaggtgaagttgttgatgcgattgaatcaaaatttggttctttagatgcatttaaacaagaatttgctgatgctgcagctgggcgcttcggttcaggctgggcatggttagttgttaataacggagagcttgaagtaacttcaacgccaaaccaggaaaatccattaatggaaggcaaaacaccaatcttaggat

SEQ ID NO: 95 - DNA, 26 bp, Artificial Sequence
  synthetic DNA
ccntayncntaygaygcnytngarcc

SEQ ID NO: 96 - DNA, 26 bp, Artificial Sequence
  synthetic DNA
arrtartangcrtgytcccanacrtc